(19) United States
(12) Patent Application Publication
(10) Pub. No.: US 2025/0380989 A1
(43) Pub. Date: Dec. 18, 2025

(54) SYSTEMS AND METHODS FOR DETERMINING A DEPLOYMENT LOCATION OF A MEDICAL INSTRUMENT

(71) Applicant: INTUITIVE SURGICAL OPERATIONS, INC., Sunnyvale, CA (US)

(72) Inventors: Serena H. Wong, Los Altos, CA (US); Joy Janku, Amsterdam (NL); Hui Zhang, Palo Alto, CA (US)

(21) Appl. No.: 18/878,547

(22) PCT Filed: Jun. 28, 2023
(86) PCT No.: PCT/US2023/069285

§ 371 Date: Dec. 23, 2024

Related U.S. Application Data
(60) Provisional application No. 63/356,902, filed on Jun. 29, 2022.

(51) Int. Cl. A61B 34/10; A61B 34/00

(52) U.S. Cl. CPC A61B 34/10; A61B 34/25; A61B 2034/105; A61B 2034/107; A61B 2034/252

(57) ABSTRACT

A medical system comprises a control system configured to receive first imaging data of a patient anatomy and identify an anatomical target in the patient anatomy. The control system is further configured to generate a treatment zone having a first axis. The treatment zone includes the anatomical target. The control system is further configured to determine a deployment position of an elongate device configured to receive a medical instrument for treatment of the anatomical target. The deployment position is aligned with the first axis of the treatment zone.

### CROSS-REFERENCED APPLICATIONS

This application claims priority to and the benefit of U.S. Provisional Application No. 63/356,902, filed Jun. 29, 2022, and entitled “Systems and Methods for Determining a Deployment Location of a Medical Instrument,” which is incorporated by reference herein in its entirety.

### FIELD

Examples described herein relate to systems and methods for determining a deployment location of a medical instrument, such as systems and methods for planning a treatment zone including an anatomical target in a patient anatomy to determine an optimal deployment location of a medical instrument aligned with the treatment zone.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Minimally invasive medical tools may also include imaging instruments such as endoscopic instruments. Imaging instruments provide a user with a field of view within the patient anatomy. Some minimally invasive medical tools and imaging instruments may be teleoperated or otherwise computer-assisted. These tools and instruments may be registered to image data of the patient anatomy to improve performance.

### SUMMARY

The following presents a simplified summary of various examples described herein and is not intended to identify key or critical elements or to delineate the scope of the claims.

Consistent with some examples, a medical system is provided. The medical system includes a control system configured to receive first imaging data of a patient anatomy, identify an anatomical target in the patient anatomy, and generate a treatment zone having a first axis. The treatment zone includes the anatomical target. The control system is further configured to determine a deployment position of an elongate device configured to receive a medical instrument for treatment of the anatomical target. The deployment position is aligned with the first axis of the treatment zone.

Consistent with some examples, a medical system is provided. The medical system includes an elongate device configured to receive a medical instrument within the elongate device. The medical system further includes a control system configured to receive first imaging data of a patient anatomy, identify an anatomical target in the patient anatomy, and generate a treatment zone having a first axis. The treatment zone includes the anatomical target. The control system is further configured to determine a deployment range of the elongate device based on the first axis of the clinical treatment zone.

Consistent with some examples, a medical system is provided. The medical system includes an elongate device configured to receive a medical instrument within the elongate device. The medical system further includes a control system configured to receive first imaging data of a patient anatomy, identify an anatomical target in the patient anatomy, determine a deployment range of the elongate device, and generate a composite treatment zone based on the deployment range. The composite treatment zone includes the anatomical target.

Consistent with some examples, a method is provided. The method includes receiving first imaging data of a patient anatomy, receiving information identifying an anatomical target in the patient anatomy, generating a treatment zone including the anatomical target, and determining a first axis of the treatment zone to identify a deployment location of an elongate device. The deployment location is aligned with the first axis of the treatment zone.

Consistent with some examples, a medical system is provided. The medical system includes a display system, an elongate device, a medical instrument configured to extend within the elongate device, and a control system communicatively coupled to the display system. The control system is configured to display a graphical user interface via the display system. The graphical user interface includes a virtual navigation view. The control system is further configured to display an image of the elongate device in the virtual navigation view, display an anatomical target in the virtual navigation view, and generate a treatment zone having a first axis. The treatment zone includes the anatomical target. The control system is further configured to determine a deployment location of the elongate device. The deployment location is aligned with the first axis of the treatment zone.

Other examples include corresponding computer systems, apparatus, and computer programs recorded on one or more computer storage devices, each configured to perform the actions of the methods.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a patient anatomy including an anatomical target according to some examples.

FIGS. 2A and 2B illustrate a graphical user interface in a planning or navigation mode according to some examples.

FIGS. 3A-3C illustrate a graphical user interface in a planning or navigation mode according to some examples.

FIG. 4 illustrates a system including a controls diagram for creating a treatment plan according to some examples.

FIG. 4A illustrates a method for determining a clinical treatment zone according to some examples.

FIG. 4B illustrates a method for determining one or more ablation zones that cover an optimal treatment zone according to some examples.

FIGS. 5A and 5B illustrate alternative methods for determining one or more composite treatment zones according to some examples.

FIG. 6 illustrates an image of a medical instrument registered to an anatomic model according to some examples

FIG. 7 illustrates a graphical user interface displaying intraoperative external imaging data and a path view according to some examples.

FIG. 8A illustrates a graphical user interface for adjusting a size or shape of one or more ablation zones according to some examples.

FIGS. 8B and 8C illustrate a graphical user interface for adjusting a position of a medical tool according to some examples.

FIG. 9 illustrates an optional method for planning a medical procedure and navigating a medical instrument during the medical procedure according to some examples.

FIG. 10 is a simplified diagram of a robotic-assisted medical system according to some examples.

FIG. 11 is a simplified diagram of a medical instrument system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

The techniques disclosed in this document may be used to enhance the workflow processes of minimally invasive procedures, such as ablation procedures. In some examples, an optimal deployment location of a medical instrument may be determined to ensure complete coverage of a clinical treatment zone is provided by the medical instrument. In some examples, image data produced by one or more intraoperative external imaging devices may be utilized to refine locations of the medical instrument, a tool, an anatomic structure, and/or a target in a model constructed from preoperative imaging.

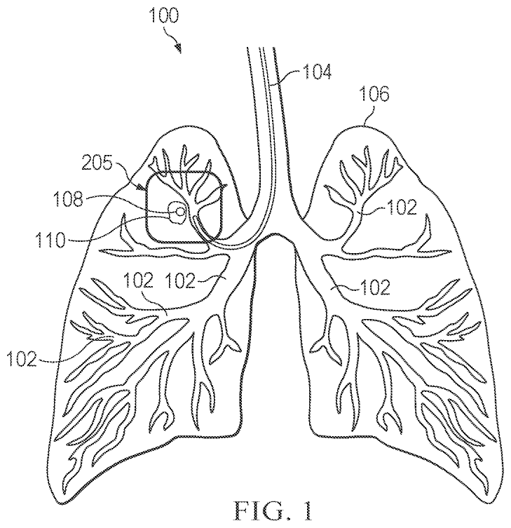

FIG. 1 is an illustration of a patient anatomy 100, specifically a patient's lungs 106 including airways 102 and an anatomical target 108, such as a lesion or nodule of interest with a margin 110 surrounding the anatomical target 108. The patient anatomy 100 includes surrounding anatomical structures, such as blood vessels, organs, pleura, fissures, etc. (not shown) proximate the lungs 106. A medical instrument 104 may be navigated through the airways 102 to the anatomical target 108 to perform a medical procedure such as diagnosis, biopsy, treatment, identification, examination, etc. For treatment of the anatomical target 108, treatment planning can be performed to optimize ideal delivery of treatment devices to provide optimal treatment.

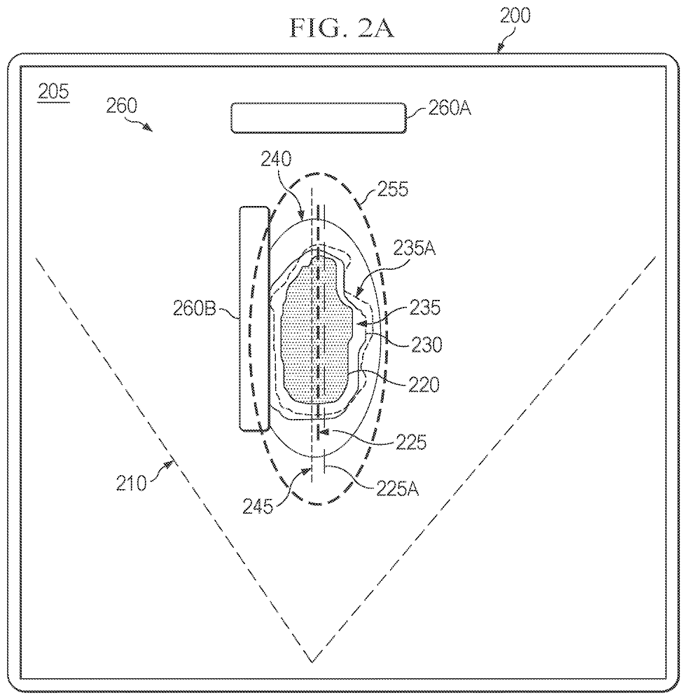

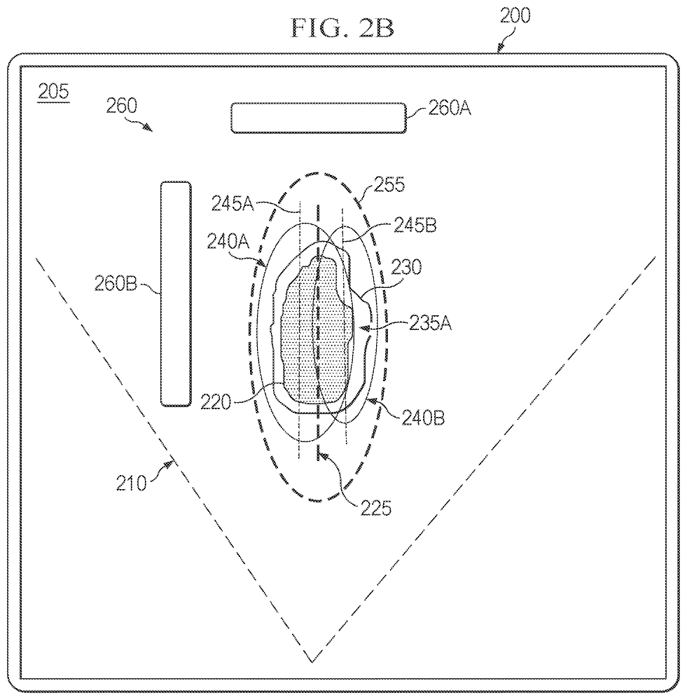

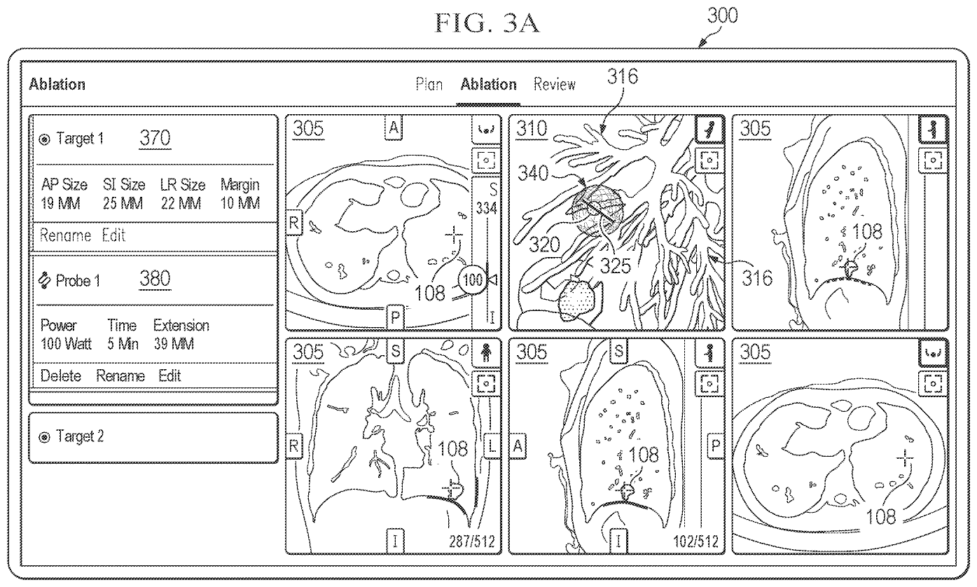

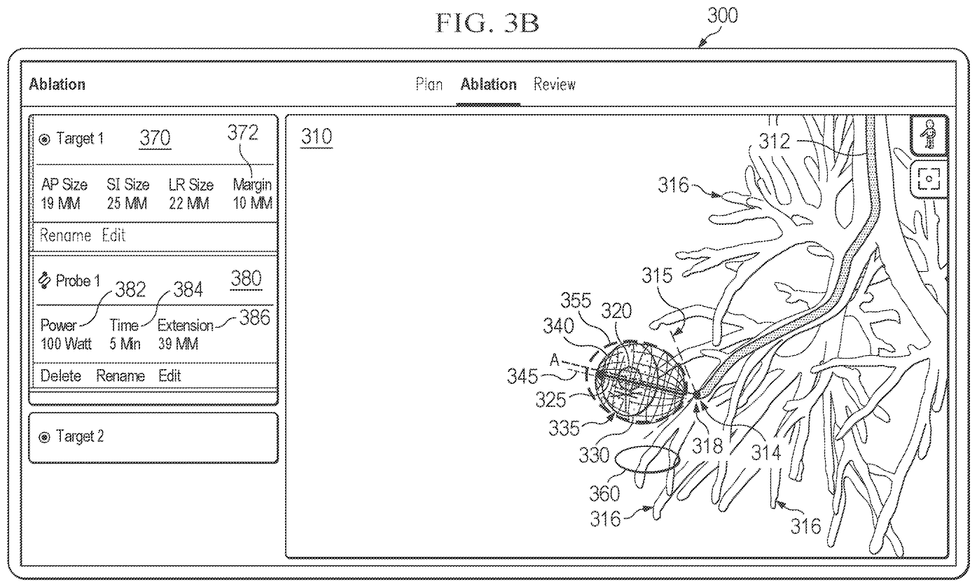

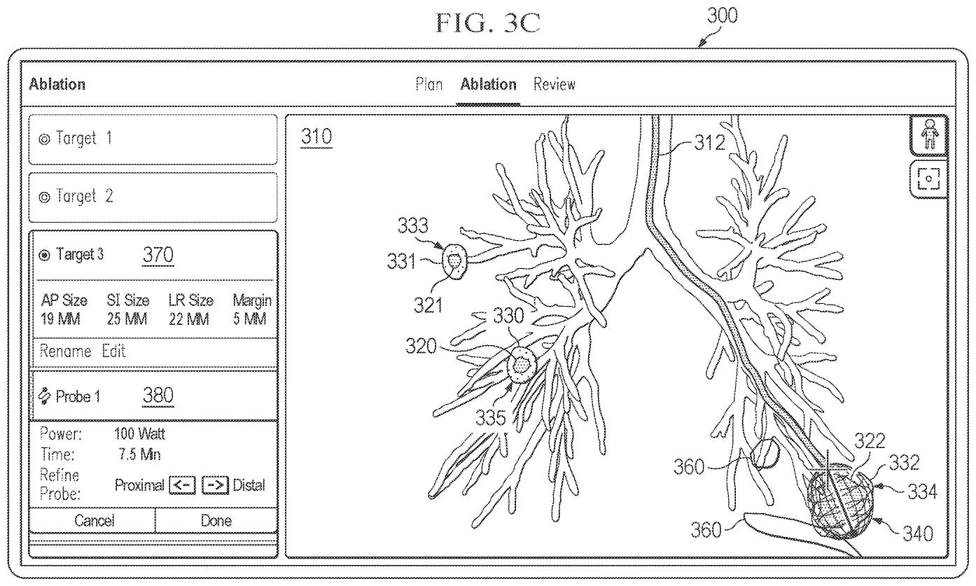

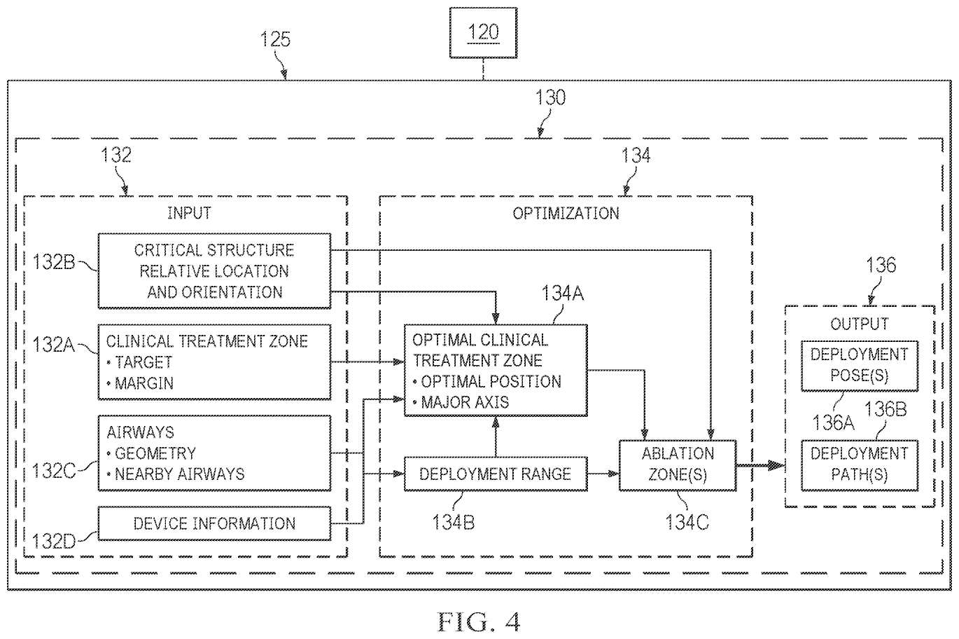

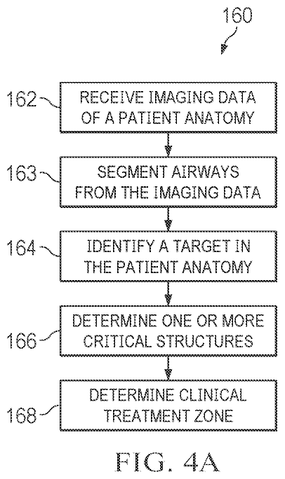

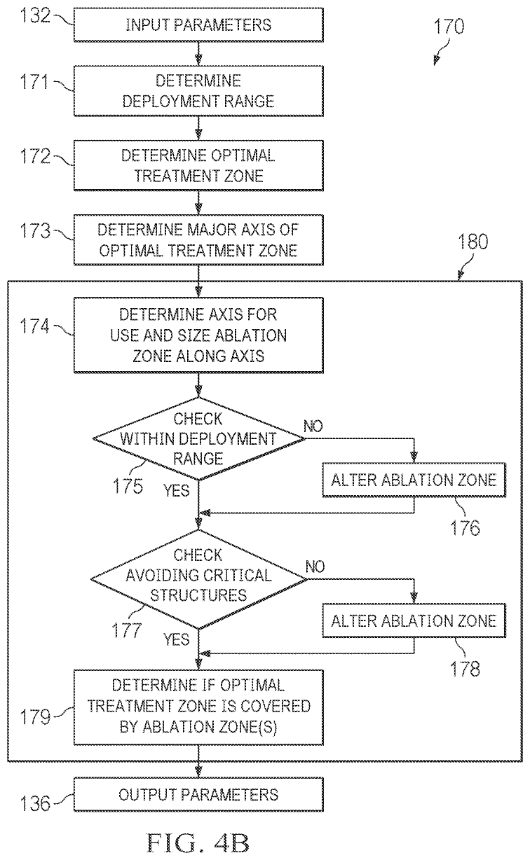

FIGS. 2A-3C illustrate an example of a graphical user interface (GUI) 300 in a planning or navigation mode during the performance of a method 160 (FIG. 4A) according to some examples. With reference to FIGS. 2A and 2B, a display system 200 may display an image 205, which represents an area (as illustrated in FIG. 1) surrounding an anatomical target 220 (e.g., the target 108), a margin 230 (e.g., the margin 110), and critical structures. In some examples, the image 205 displays a deployment range 210 for a medical instrument, such as the medical instrument 104. The image 205 may further display the target 220, the margin 230, a clinical treatment zone 235, a major axis 225 of the clinical treatment zone 235, an ablation zone 240, a major axis 245 of the ablation zone 240, and one or more critical structures 260 (e.g., the heart, blood vessels, and/or airways). In some examples, the image 205 may also display one or more anatomical structures, such as fissures or pleura in the lungs.

With reference to FIGS. 3A-3C, the GUI 300 (which may be displayed on a display, e.g., the display 200) includes a virtual navigation view 310. The virtual navigation view 310 may illustrate a medical instrument 312 (e.g., the medical instrument 104), one or more anatomical passageways 316, an anatomical target 320 (e.g., the target 108), a deployment range 315 for the medical instrument 312, a longitudinal axis A of the medical instrument 312, a margin 330, a clinical treatment zone 335 (e.g., the clinical treatment zone 235), a major axis 325 of the clinical treatment zone 335, an ablation zone 340, a major axis 345 of the ablation zone 340, and a probe safety margin 355.

In some examples, the different zones, axes, and features discussed above may be displayed in the images with different colors. For example, the target may be shown in one color (e.g., blue), and the margin may be shown in another color (e.g., yellow). Any other colors may be used to display the different zones, axes, and features discussed above. In some examples, the different zones, axes, and features discussed above may be displayed in the image with different patterns, symbols, reference numbers, or any other graphical identifiers.

Although illustrative arrangements of views are depicted in FIGS. 2A-3C, it is to be understood that the GUI 300 may display any number of views, in any arrangement, and/or on any number of screens. In some examples, the number of concurrently displayed views may be varied by opening and closing views, minimizing and maximizing views, moving views between a foreground and a background of the GUI 300, switching between screens, and/or otherwise fully or partially obscuring views. Similarly, the arrangement of the views—including their size, shape, orientation, ordering (in a case of overlapping views), and/or the like—may vary and/or may be user-configurable. While being described above in the context of planning a procedure, the GUI 300 may be displayed during the planning stage, a navigation stage, or both the planning and navigation stages of a procedure.

In some examples, a control system or processing system may be used to identify and determine an anatomical target, margin, critical structures, various zones, and deployment ranges used to determine optimal deployment locations to create an ablation plan to optimize ablation treatment.

FIG. 4 illustrates a system including a display system 120 (e.g., display system 200/300) coupled to a control system 125, or processing system, which includes one or more processors. The control system 125 can perform processes according to a controls diagram 130 for an optimization framework used to create a treatment plan, according to some examples. The optimization processes described herein are illustrated as a set of parameters used to execute processes that may be performed in a sequential or simultaneous order. One or more of the illustrated processes may be omitted in some examples. Additionally, one or more processes that are not expressly illustrated may be included before, after, in between, or as part of the illustrated processes. In some examples, one or more of the processes may be implemented, at least in part, by a control system (e.g., the control system 125) executing code stored on non-transitory, tangible, machine-readable media that when run by one or more processors (e.g., the processors of a control system, such as the control system 125) may cause the one or more processors to perform one or more of the processes.

As shown in FIG. 4, the controls diagram 130 includes a set of initial input parameters 132, a set of optimization parameters 134, and a set of output parameters 136. The initial input parameters 132 may include a clinical treatment zone 132A (e.g., the clinical treatment zone 235), critical structures 132B (e.g., the critical structures 260), anatomical airways 132C (e.g., the anatomical airways 102), and device information 132D (e.g., information for the medical instrument 104). Once the input parameters 132 have been defined, the control system 125 may use the inputs to determine the optimization parameters 134. The optimization parameters 134 may include an optimal clinical treatment zone 134A, a deployment range 134B (e.g., the deployment range 210) for the device 104, and one or more ablation zones 134C (e.g., the ablation zone 240). Based on the optimization parameters 134, the control system 125 may determine one or more of the output parameters 136. The output parameters 136 may include one or more deployment poses 136A and one or more deployment paths 136B.

FIG. 4A is a flowchart illustrating a method 160 for determining a clinical treatment zone, according to some examples. The method 160 and other methods described herein are illustrated as a set of operations or processes that may be performed in the same or in a different order than the order shown in the figure. One or more of the illustrated processes may be omitted in some examples of the method. Additionally, one or more processes that are not expressly illustrated in the flowcharts may be included before, after, in between, or as part of the illustrated processes. In some examples, one or more of the processes of the flowcharts may be implemented, at least in part, by a control system executing code stored on non-transitory, tangible, machine-readable media that when run by one or more processors (e.g., the processors of a control system) may cause the one or more processors to perform one or more of the processes.

At a process 162, imaging data of a patient anatomy is received. The imaging data may be received at the control system 125. The imaging data may be preoperative imaging data. For example, a CT scan, which may be a cone beam CT scan, of the patient anatomy may be performed with a CT scanner, and the CT image data may be received by the control system 125. Alternatively, the preoperative imaging data may be received from other types of imaging systems including magnetic resonance imaging (MRI) systems, fluoroscopy systems, or any other suitable method for obtaining dimensions of anatomic structures. At a process 163, anatomical airways are segmented from the imaging data of the patient anatomy. The anatomical airways may be identified in the imaging data of the patient anatomy. In some examples, a model of the anatomical airways may be generated based on the segmented anatomical airways. Various segmentation functions may be used to create the model. In some examples, the segmentation function may rely on a set of seed points selected by a user. For example, a clinician may review the imaging data of the patient anatomy and manually select seed points that identify one or more anatomical airways within the patient anatomy. Additionally or alternatively, one or more processors (e.g., one or more image processors) of the control system 125 may identify the anatomical airways in the patient anatomy and set seed points for the segmentation function based on the identified anatomical airways.

At a process 164, a target (e.g., the target 108) may be identified in the patient anatomy. In some examples, the target 108 is identified in the preoperative imaging data of the patient anatomy as illustrated in FIG. 3A. For example, the target 108 may be identified in CT slides as shown in the windows 305 as a region of interest for investigation and/or treatment. The target 108 may be automatically identified by the control system 125 and confirmed by a user, or the target 108 may be visually identified by the user and manually selected or indicated in the 3D model 106, for example, via the display system 100. In some examples, the control system 125 and/or a user may determine one or more parameters of the target 108 based on the imaging data. For example, the major axis, the density, the hardness, and/or any other physical property of the target 108 may be determined.

At a process 166, critical structures near the target 108, such as organs (e.g., the heart), blood vessels, and/or nearby anatomical passageways (e.g., airways of a lung) may also be identified in the imaging data. Additionally or alternatively, tissue characteristics of the patient anatomy (e.g., emphysema percentage surrounding the target, fibrosis, necrotic tissue, etc.) and/or other anatomical structures, such as fissures or pleura of a lung, may be identified in the imaging data. In some examples, a safety score for one or more of the critical structures may be determined. The safety score may be determined based on size of the structure, proximity to the target 108, potential heat sink effect during ablation, and/or any one or more additional similar factors. In some examples, a target region around the target 108 may be determined. The target region may be a sphere or ellipsoid centered on the target 108. The target region may have a radius of 5 cm, 10 cm, 15 cm, or any other suitable radius.

In some examples, a 3D model of the anatomic structures (e.g., the anatomic model 106) may be constructed from the preoperative imaging data by the control system 125 as illustrated in the window 310 of FIGS. 3A-3C. Any one or more of the target 108, the critical structures (e.g., organs, blood vessels, nearby anatomical passageways, etc.), the anatomical passageways (e.g., additional anatomical passageways within the full lung), the tissue characteristics, or the anatomical structures may be identified in the 3D model 106 and/or in the preoperative imaging data from which it was constructed.

At a process 168, a clinical treatment zone 235 is determined. In some examples, the clinical treatment zone 235 represents the area within the patient anatomy where a treatment should be applied by a medical instrument, such as the medical instrument 104. For example, the clinical treatment zone 235 may be the area within the patient anatomy that should be ablated by an ablation probe, which may be extended from the medical instrument 104. As shown in FIGS. 2A and 2B, the clinical treatment zone 235 can be defined by the target 108 and the surrounding margin 110.

The margin 230 surrounds the target 220 to act as a safety margin to ensure that all diseased cells are ablated. The margin may also be sized and shaped to account for portions of the target 220 that may not be visible in the image 205. The margin 230 may alternatively or additionally be sized to account for any computational error and/or execution error that may be present when determining the size and shape of the target 220. The clinical treatment zone 235 includes the area covered by the target 220 and the margin 230.

In some examples, the margin 230 may be uniform around the entirety of the target 220. For example, the margin 230 may provide a 5 mm buffer around the target 220. The margin 230 may provide a buffer of any other size, such as 3 mm, 8 mm, 10 mm, or any other size that may encompass portions of the target 220 that may not be visible in the image 205. In some examples, one or more portions of the margin 230 may be non-uniform around the entirety of the target 220 and may be different lengths at different parts of the target 220. For example, one portion of the margin 230 may provide a 5 mm buffer and another portion of the margin 230 may provide a 3 mm buffer. The margin 230 may be non-uniform due to, for example, the type of target 220, the proximity of the critical structures 260, the proximity of anatomical structures, or any other similar factor.

In some examples, the size of the margin 230 may be automatically set and/or adjusted by a control system (e.g., the control system 125) based on the type of target 220 to be ablated. For example, the control system may retrieve information from a target database that includes dimensions and other physical characteristics of different types of anatomical targets. Additionally or alternatively, an image processor of the control system may perform image analysis of the image 205 to determine the size and shape of the target 220.

In some examples, the size of the margin 230 may be set and/or adjusted by a user. The size and/or shape of the margin 230 may be adjusted or altered to account for patient movement (e.g., respiratory movement or circulatory movement) and/or CT to body divergence. For example, in reference to FIGS. 3A-3C, the control system may receive a user input via the target menu 370 of the GUI 300 that adjusts the margin size icon 372. In some examples, a table or menu (not shown) may be displayed in the GUI 300 that includes a list of spheres and/or ellipses with pre-specified sizes. The control system may receive a user input selecting one or more of the spheres and/or ellipses. The selected spheres and/or ellipses may be displayed in the virtual navigation view 310. In some examples, the control system may receive the user input via the GUI 300. Additionally or alternatively, the user input may be numerical values defining the distance the margin 330 should expand beyond the outer edges of the target 320.

In some examples, the control system adjusts the margin 330. The margin 330 may be displayed in the virtual navigation view 310 before or after any adjustments are made to the size/shape of the margin 330. In some examples, the adjustments made to the margin 330 may be shown as an animation in the virtual navigation view 310 to allow the user to visualize the changes made to the margin 330. In examples when the margin 330 is user-adjustable, the control system may receive one or more user inputs via a GUI, such as the GUI 300, that indicate how the margin 330 is to be adjusted. In some examples, the GUI 300 includes a touchscreen. In such examples, control system may receive a user input via the touchscreen indicating how the margin 330 is to be adjusted.

Referring back to the input parameters 132 of FIG. 4, in some examples the control system 125 may receive a user input via the GUI 300 indicating the type of treatment device (e.g., the medical instrument 104) to be used for a procedure. In some examples, the control system 125 may receive a user input indicating ablation zone information (e.g., ablation zone size, orientation, position, etc.) with reference to one or more physical properties of the treatment device (e.g., bending stiffness, maximum bend angle, outer diameter, etc.). Alternatively, the user can input medical procedure information and the system may recommend a type of treatment device based on the medical procedure. Information specific to the device can in input into the control system or saved and accessed through internal memory. In some examples, the user may input clinical constraints including a maximum overall procedure time, a maximum allowable energy delivery time per treatment, a maximum allowable treatment power level, etc.

Using the input parameters 132, the optimization parameters 134 including the optimal clinical treatment zone 134A, the deployment range 134B (e.g., the deployment range 210), and one or more ablation zones 134C (e.g., the ablation zone 240) can be determined. FIG. 4B illustrates one example method 170 for determining the optimization parameters 134 from input parameters 132.

Referring to FIG. 4B, at a process 171, one or more deployment ranges (e.g., deployment range 210/315) are determined based on airway geometry and device information determined based on the input parameters 132. The deployment range 210, may be a boundary within which a medical instrument (e.g., the medical instrument 104) may approach an anatomical target, e.g., the target 108, establishing areas which can be reached by the medical instrument. The deployment range may include a range in three-dimensional space of insertion depths for the medical instrument as well as a range of angles from which the medical instrument may approach the anatomical target. In one example, a deployment range 210 may be established by determining a deployment pose, which may include a deployment location and a deployment orientation. The deployment location is a parked location of a catheter (e.g., the medical instrument 104) distal end or distal end section from which an instrument, such as an ablation probe, is extended to perform treatment. The deployment orientation is a pointing direction of a catheter (e.g., the medical instrument 104) distal end section from which an instrument, such as an ablation probe, is extended to perform treatment. Further description of the deployment pose will be provided below.

The deployment range 210 may indicate the boundaries within which the medical instrument may approach an anatomical target 220 (e.g., the target 108). In some examples, the deployment range 210 may be restricted based on mechanical properties of the medical instrument (e.g., bending stiffness, maximum bend angle, or outer diameter) as defined by the type of medical instrument/medical probe and/or physical properties of the anatomical passageways (e.g., maximum bend angle or diameter). In some examples, the medical instrument 104 includes a treatment instrument such as an ablation probe. In other examples, the medical instrument 104 may include a delivery instrument such as a delivery catheter and a treatment instrument delivered through a lumen of the delivery catheter. In such cases, the combined mechanical properties of both the delivery catheter and the medical instrument could affect the deployment range.

The boundaries and size of the deployment range 210 may be based on one or more constraints imposed by the anatomical passageway(s) in which the medical instrument is positioned. For example, the medical instrument may be unable to be oriented outside of a range of angles towards a target when positioned in an anatomical passageway which includes a bend that has a bend radius that is smaller than the maximum bend radius of the medical instrument or if the diameter of the passageway is smaller than the outer diameter of the medical instrument or too small to allow the medical instrument to bend towards the target. Other physical characteristics of the passageway may impact the ability of the medical instrument to traverse the passageway. One or more of these constraints may limit the available deployment locations of the medical instrument. The deployment range 210 is sized to include some or all of the available deployment locations where the medical instrument may access the target 220.

As shown in FIG. 3B, a deployment range 315, which is the same as the deployment range 210 discussed above, may be established by initially aligning a longitudinal axis A of the medical instrument 312 with the major axis 325 of the clinical treatment zone 335 such that the distal end 314 of the medical instrument 312 is pointed along the major axis 325. The deployment range 315 may include all possible deployment orientations established by articulating the medical instrument 312 from the initial location where such an alignment between the medical instrument 312 and the major axis 325 of the clinical treatment zone 335 establishing a cone or three-dimensional fan creating the deployment range 315.

In some examples (not shown), multiple deployment ranges may be determined when multiple paths to the target 320 are possible—e.g., when the medical instrument 312 may approach the target 320 through different sets of multiple anatomical passageways 316. For example, the deployment range 315 may be determined based on which set of anatomical passageways 316 the medical instrument 312 is able to traverse to reach the target 320. If the target 108 is in-between two branches, one deployment range can be established if the medical instrument 104 approaches the target 108 from one branch through one path along a set of anatomical passageways, while a second deployment range can be established if the medical instrument 104 approaches the target 108 from a different branch through a different path along a set of at least some different anatomical passageways. Each of the available paths through different anatomical passageways 102 includes a maximum bend angle that is less than the maximum bend radius of the medical instrument 104 but based on the differing geometries, the different paths provide for different angles of deployment ranges. The deployment range 315 may exclude paths that do not allow for the longitudinal axis A of the medical instrument 312 to be aligned with the major axis 325 of the clinical treatment zone 335.

Referring back to FIG. 4B, at a process 172, an optimal treatment zone may be determined. The optimal treatment zone can be determined by adjusting the clinical treatment zone (e.g., shape may be altered, location may be shifted, zone may be rotated, etc.) to account for critical structures (e.g., blood vessels, nearby airways, organs, etc.). For example, as illustrated in FIG. 2A, the clinical treatment zone 235 is shown overlapping with the critical structure 260 resulting in a shifting of the clinical treatment zone 235 to form an optimal treatment zone 235A. Additionally, in some examples where the margin 230 can remain an acceptable size for clinical treatment, the size and shape of the clinical treatment zone 235 may be altered (not shown) to avoid one or more critical structures, such as the heart, blood vessels, nearby airways, pleura of the lungs, fissures of the lungs, and the boundary of the lung. For example, FIG. 3B illustrates a critical structure 360 near the target 320. The critical structure 360 may represent a boundary of the lung. In some examples, only critical structures that are located near the target 320 are displayed in the virtual navigation view 310. For example, critical structures within 15 cm of the target 320 may be displayed. Any other proximity range (e.g., 5 cm, 10 cm, or 20 cm) may be used to determine which critical structures, if any, are displayed.

In some examples, the clinical treatment zone 335 may be adjusted or shifted to account for the critical structure(s) 360. For example, the major axis 325 of the clinical treatment zone 335 may be shifted laterally away from the critical structure 360. In examples when the critical structure 360 is a blood vessel, shifting the major axis 325 away from the blood vessel may avoid hemoptysis of the blood vessel. In other examples where the critical structure 360 is a nearby airway, shifting the major axis 325 away from the airway may avoid unwanted cooling effects caused by airflow through the airway. In some examples, the shifted major axis 325 may be generally perpendicular to a critical structure 360. In some examples, the clinical treatment zone 335 may be rotated based on the position of the critical structure 360.

Referring back to the method 170 of FIG. 4B, at a process 173, after the clinical treatment zone 235 is adjusted to create the optimal treatment zone 235A, a major axis 225A of the optimal treatment zone 235A is determined, as illustrated in FIG. 2A. The method 170 may then move to a process 180 where one or more ablation zones covering the optimal treatment zone 235A may be determined.

The ablation zone 240 represents the predicted area that will be treated (e.g., ablated) by a medical instrument, such as an ablation probe, during a single treatment procedure, such as a single delivery of energy for an uninterrupted duration of time during an ablation procedure. The area covered by the ablation zone 240 may be an ablation region. An additional uninterrupted delivery of energy at a different time and/or a different location can create an additional separate ablation zone covering a separate ablation region. In some cases, separate ablation zones and ablation regions may be used, as will be described in more detail below.

The predicted size and shape of the ablation zone 240 is based on the design construction of the ablation probe, an amount of energy applied to the probe, a duration of time the energy is applied to the probe, and one or more tissue characteristics of the target 220 within which the ablation probe is deployed. The tissue characteristics of the target 220 may include density, hardness, an emphysema percentage surrounding the target 220, fibrosis, necrotic tissue, proximity to critical structures 260, or any other physical characteristic of the target 220 or of the anatomy surrounding the target 220. In some examples, the tissue characteristics are determined at the process 168 of the method 160 in FIG. 4A. Accordingly, ablation zones of various shapes and sizes may be predicted by altering the duration of power and energy delivery at different ablation probe transducer locations within the anatomy with different tissue characteristics.

In some examples, the optimal treatment zone 235A can be fully covered by one ablation zone. FIGS. 3B and 3C illustrate one ablation zone 340. The ablation zone 340 may be sized to be as small as possible while still fully covering the clinical treatment zone 335. Reducing the size of the ablation zone 340 may limit the effects of the ablation treatment on the anatomy surrounding the clinical treatment zone 335, such as any critical structures that may be near the target 320.

As shown in FIGS. 3A-3C, the ablation zone 340 is three-dimensional. The size and shape of the ablation zone 340 may be determined based on ablation parameters, such as power, time, and ablation probe insertion distance. If more than one ablation zone is needed to fully ablate the clinical treatment zone 335 (as will be described in more detail below), the ablation parameters may differ for each ablation zone that is needed. In some examples, some or all of the ablation parameters may be adjusted by the user. For example, the control system may receive one or more user inputs via the probe menu 380 of the GUI 300 that adjusts one or more of the probe power icon 382, the treatment time icon 384, and the probe insertion distance icon 386. Additionally or alternatively, the ablation parameters may be adjusted by the control system.

In some examples, a maximum size of the ablation zone 340 may be determined based on the design of the ablation probe and characteristics of the patient anatomy near the target 320. For example, the control system may include or have access to a database including characteristics for one or more types of different ablation probes, which may be from different manufacturers or vendors. Each ablation probe includes a set of physical characteristics, such as maximum length, diameter, maximum available power, or other physical characteristics. Based on these characteristics, each ablation probe has a maximum ablation zone size that each probe can generate. In some examples, a table of the ablation zone sizes may be displayed in the GUI 300 showing various ablation zone sizes based on input characteristics such as input power, input duration, time, etc. The control system may select or may receive a user input via the GUI 300 selecting the type of ablation probe that will be used for the treatment procedure as previously described and access (and in some examples display) the table for the selected ablation probe. The GUI 300 may display the ablation zone 340 in the virtual navigation view 310, and the ablation zone 340 may be sized based on the maximum ablation zone size for the selected ablation probe. The size and/or shape of the ablation zone 340 may then be further adjusted by the control system and/or by the user by altering the power settings, duration settings, or location of the ablation probe, which may alter the center of the ablation zone 340.

In some examples, more than one energy delivery treatment (e.g., an ablation treatment) is needed. Accordingly, the number, location, and orientation of multiple ablation zones may be determined. The ablation zones may be sized to minimize the total number of ablation zones needed to cover the clinical treatment zone 335. This may reduce the number of ablation treatments needed to fully treat the target 320 and the margin 330 and may reduce the amount of healthy tissue that is ablated during the ablation treatments. In some examples, to obtain ablation zones of different sizes, the ablation treatments may have different power outputs and durations, and the ablation probe may be inserted to different insertion distances for each ablation treatment.

Referring back to FIG. 4B, process 180 is an example of an iterative process to determine one or more ablation zones to optimally cover the optimal treatment zone 235A. At process 174, an ablation zone 240 may be determined along an axis for use. Initially, when executing process 174 after process 173, the axis for use is the major axis of the optimal treatment zone from process 173. The ablation zone 240 may be sized to cover the optimal clinical treatment zone 235 as shown in the image 205. In some examples, the major axis 245 of the ablation zone 240 may be coincident with the major axis 225A of the optimal treatment zone 235A. In some examples, the major axis 245 of the ablation zone 240 may be parallel or substantially parallel, but not coincident, with the major axis 225 of the clinical treatment zone 235. In some examples, the major axis 225 of the clinical treatment zone 235 may be coincident with a major axis of the target 220. In some examples, the major axis 225 of the clinical treatment zone 235 may be parallel or substantially parallel, but not coincident, with the major axis of the target 220.

Referring back to FIG. 4B, at process 175 the ablation zone 240 is verified to fit within the deployment range 215. For example, the control system and/or a user may verify that it is possible to deliver an ablation probe to create the ablation zone 240 determined at process 174. If it is determined that the ablation zone 240 fits within the deployment range 315, then the method 170 can proceed to process 177. If it is determined that the ablation zone 240 does not fit within the deployment range, then the method 170 proceeds to process 176 where the ablation zone 240 can be altered to fit within the deployment range 215. The location and orientation of the ablation zone 240 may be shifted and rotated respectively to adjust the location and orientation of the ablation zone 240 according to the deployment range 215. In some examples, an updated location or orientation may require that the ablation zone 240 be resized to continue to provide maximum coverage of the optimal treatment zone 235A. The control system may limit the maximum size of the ablation zone 240 during re-sizing based on the new location of the ablation zone 240 (e.g., updated proximity to critical structures and tissue properties affecting ablation zone size) as previously described. Once the ablation zone 240 has been updated to conform within the deployment range 215, the method 170 may proceed to process 177.

At process 177, the ablation zone 240 is verified to avoid critical structure(s) 260. If the ablation zone 240 avoids critical structures 260, the method 170 can move on to process 179. If the ablation zone 240 overlaps with critical structures 260, the method 170 moves to process 178 where the ablation zone 240 is altered to avoid the critical structures 260. For example, the ablation zone parameters shown in the probe menu 280 may be adjusted based on the proximity of the ablation zone 240 to one or more critical structures, such as the critical structure 260A. In some examples, if an ablation is being performed near one or more blood vessels and/or nearby airways, a heat sink or cooling effect may be created by blood flow through the blood vessels and/or airflow through nearby airways. The blood vessels and airways may act as a heat sink/cooling mechanism and pull some heat from the ablation probe that otherwise would have been applied to the target 220 and the margin 230. In such examples, a treatment parameter such as the power output by the ablation probe, the duration of the ablation treatment, or both may need to be increased to ensure the target 220 and the margin 230 are fully treated. In some examples, the ablation zone parameters may be adjusted based on tissue characteristics of the patient anatomy within the ablation zone 240. For example, the density, hardness, material composition, or any other characteristics of the tissue within the ablation zone 240 may affect how much power should be output by the ablation probe and/or the duration of the ablation treatment to fully treat the target 220 and the margin 230. In some examples, the ablation zone 240 may be rotated based on the proximity of the ablation zone 240 to one or more critical structure(s) 260.

Once the ablation zone has been sufficiently altered to avoid critical structures, the method 170 can move on to process 179 to determine if the ablation zone 240 completely covers the optimal treatment zone 235A. If the optimal treatment zone 235A is fully covered by the ablation zone 240 as illustrated in FIG. 2A, then the method 170 may move on to determine the output parameters 136. However, if the ablation zone 240 does not fully cover the optimal treatment zone 235A, the method 170 returns to process 174 to determine additional ablation zones based on an updated axis for use.

As shown in FIG. 2B, in some examples, more than one ablation zone 240A, 240B may be needed to cover the entire margin 230. For example, due to the physical characteristics of the target 220, the anatomy surrounding the target 220, procedure time constraints, and/or the limitations of the ablation probe, more than one ablative treatment may be needed to fully treat the optimal treatment zone 235A. Depending on the proximity of the critical structures 260, for example, one ablation zone 240 that covers the entire optimal treatment zone 235A may require a certain amount of energy or time to complete the ablative treatment that may cause damage to the surrounding critical structures 260. To avoid this damage, the size of the ablation zone 240 may be reduced and/or the shape of the ablation zone 240 may be adjusted or altered. For example, the treatment settings may be adjusted to provide less energy within less time. In some examples, the orientation and/or insertion depth of the ablation probe may be adjusted, which may modify the size and/or shape of the ablation zone 240. For example, when the ablation probe is deployed from the medical instrument 104, the ablation probe may remain aligned with a longitudinal axis of the medical instrument 104. When the longitudinal axis of the medical instrument 104 is aligned with the major axis of an additional ablation zone and the ablation probe is deployed, the ablation probe may also be aligned with the major axis of the additional ablation zone. Additionally or alternatively, the ablation probe may be realigned as needed to align with the major axis of each additional ablation zone. The distal end of the medical instrument 104 may remain stationary as the ablation probe bends to be realigned with the major axis of each additional ablation zone. A separate ablation treatment may be performed when the ablation probe is aligned with the major axis of each additional ablation zone.

In another example, probe design constraints, proximity to critical structures like blood vessels acting as heat sinks or airways acting as cooling mechanisms, and tissue type requiring higher power/duration may result in a smaller maximum ablation zone which does not fully treat the optimal treatment zone. Thus, if the smaller ablation zone, such as the ablation zone 240A, does not surround the entire optimal treatment zone 235A, an additional ablation zone 240B may be needed to fully treat the optimal treatment zone 235A. Accordingly, with reference to FIG. 4B, the method 170 may progress to process 174, which includes both determining a shifted axis and a size and shape of the additional ablation zone along the shifted axis. As shown in FIG. 2B, the ablation zone 240A includes a major axis 245A, and the ablation zone 240B includes a major axis 245B. In one example, the ablation zone 240A may be shifted in one direction from the major axis of the optimal treatment zone and an additional ablation zone may be determined by identifying an additional axis shifted in an opposite direction from the major axis of the optimal treatment zone as illustrated in FIG. 2B. As illustrated, one or both of the major axes 245A, 245B may be parallel or substantially parallel with the major axis 225 of the optimal treatment zone 235A. In some examples, one or both of the major axes 245A, 245B are not parallel or substantially parallel with the major axis 225 of the optimal treatment zone 235A. For example, one or both of the major axes 245A, 245B may be perpendicular or substantially perpendicular with the major axis 225.

Since the ablation zones have been shifted, proximity to critical structures like blood vessels/airways and proximity to variable tissue type may have changed, resulting in a change in ablation zone size. Accordingly, new sizes and shapes of ablation zones may be determined based on new locations. As previously described, power settings and energy application durations may be altered to adjust the size and shape of each of the ablation zones 240A and 240B. Thus, the method 170 may continue through processes 175-179, altering power, duration, location, and orientation of ablation zones until both ablation zones 240A and 240B are within the deployment range 215 and are avoiding critical structures. At process 179, if the ablation zones 240A and 240B do not collectively cover the optimal treatment zone 235A completely, then process 180 can continue in a loop adding additional ablation zones and re-sizing and re-positioning existing ablation zones until all ablation zones fully cover the optimal treatment zone 235A. Once all ablation zones fully cover the optimal treatment zone 235A, process 180 can continue to determine the output parameters 136.

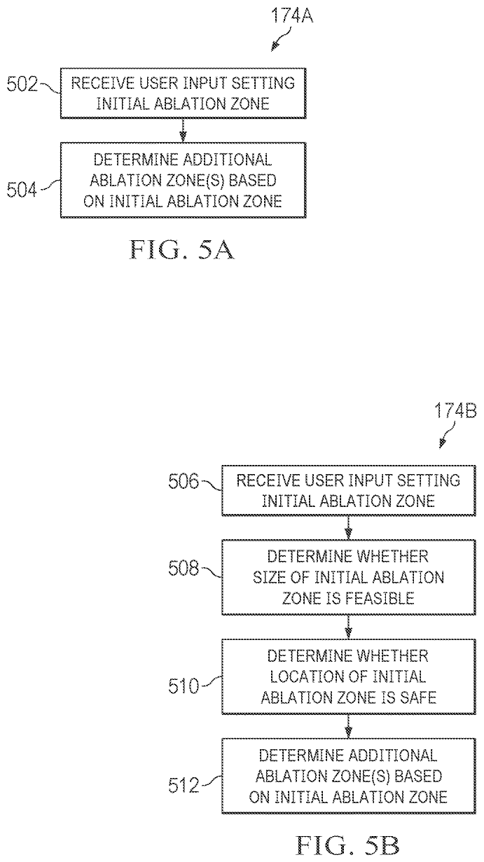

In some examples, one or more of the resizing, shifting, and/or rotating of the ablation zones may be set by a user and/or may be automatically performed by the control system. In that regard, FIG. 5A includes a flowchart illustrating a method 174A, which is an alternative method for determining ablation zones. FIG. 5B includes a flowchart illustrating a method 174B, which is another alternative method for determining the ablation zones. The methods 174A and 174B are described with continuing reference to FIGS. 2A, 2B, and 3A-3C. In some examples, each of the methods 174A, 174B may be used to determine where the ablation zone(s) should be placed. In some examples, either the method 174A or the method 174B may be used to determine where the ablation zone(s) should be placed.

With reference to the method 174A, at a process 502, the control system receives a user input setting an initial ablation zone, such as the ablation zone 340 in FIG. 3A. As discussed above, the control system may receive inputs from the user adjusting the size or shape of the initial ablation zone as needed. In some examples, the input from the user may be a touch input on a touchscreen of the display system 200. A graphical indicator may be displayed via the GUI 300, for example, when the touch input is received. Additionally or alternatively, the input from the user may be an input from a mouse received by the control system. A graphical indicator may be displayed via the GUI 300, for example, identifying a cursor location for the mouse. After the size and shape of the initial ablation zone is set, the control system may determine if one or more additional ablation zones are needed based on the size and shape of the initial ablation zone at a process 504.

With reference to the method 174B, at a process 506, the control system receives a user input setting an initial ablation zone, such as the ablation zone 340. As discussed above, the control system may receive inputs from the user adjusting the size or shape of the initial ablation zone as needed. After the size and shape of the initial ablation zone is set, the control system may determine whether the size of the initial ablation zone is feasible at a process 508. For example, the initial ablation zone may be sized based on the amount of power to be applied to the ablation probe. The control system may determine whether the ablation probe that will be used to perform the ablation treatment is capable of outputting the desired amount of power. If the ablation probe cannot output the desired amount of power, the control system may reduce the size of the initial ablation zone. The control system may reduce the size of the initial ablation zone automatically and/or in response to one or more user inputs. For example, the control system may receive one or more user inputs via the probe menu 380 of the GUI 300 that adjusts one or more of the probe power icon 382, the treatment time icon 384, and the probe insertion distance icon 386. Additionally or alternatively, the control system may automatically adjust one or more of the probe power, the treatment time, and the probe insertion distance. The automatic adjustment(s) may alter the values shown in the probe power icon 382, the treatment time icon 384, and/or the probe insertion distance icon 386.

At a process 510, the control system may determine whether the location of the initial ablation zone is safe. For example, the control system may determine that the initial ablation zone is placed too close to one or more critical structures 360 to safely perform the ablation treatment. The control system may adjust the size of the ablation zone and/or shift the placement of the ablation zone to provide more space between the ablation zone and the critical structure(s) 360. The control system may adjust the size of the ablation zone and/or shift the placement of the ablation zone automatically and/or in response to one or more user inputs. For example, the control system may receive one or more user inputs via the target menu 370 of the GUI 300 that adjusts the size of the ablation zone and/or shifts the placement of the ablation zone. Additionally or alternatively, the control system may automatically adjust the size of the ablation zone and/or shift the placement of the ablation zone. The automatic adjustment(s)/shift(s) may alter the values shown in the target menu 370.

After the size and shape of the initial ablation zone is set, the control system may determine if one or more additional ablation zones are needed based on the size and shape of the initial ablation zone at a process 512. In some examples, the control system may perform one or more of the processes 508 and 510 for one, some, or all of the additional ablation zones that may be needed.

In some examples, the size of the ablation zone 240 may be automatically set and/or adjusted or altered by the control system based on the size/shape of the margin 230. For example, the image processor of the control system may perform image analysis of the image 205 to determine the size and shape of the margin 230. In some examples, the size of the ablation zone 240 may be set and/or adjusted or altered by the user.

In some examples, a probe safety margin 255 as illustrated in FIGS. 2A and 2B may be provided. The probe safety margin 255 may account for potential movement or shifting of the position of the ablation probe. For example, once the probe is placed in its final position prior to ablation, the actual deployed position of the probe may slightly shift. In some examples, the shifting may be caused by patient motion. The probe safety margin 255 may be sized and shaped to surround the ablation zone(s) 240.

If the probe shifts position, the probe may more easily move in an axial direction than in a lateral direction. In some examples, the axial direction of movement of the probe may be parallel or substantially parallel with the major axis 225 of the clinical treatment zone 235. In some examples, the lateral direction of movement of the probe may be perpendicular or substantially perpendicular with the major axis 225 of the clinical treatment zone 235. As shown in FIGS. 2A and 2B, because the probe may more easily move in an axial direction than in a lateral direction, the probe safety margin 255 may be sized to be longer in the axial direction than in the lateral direction. This may allow for more potential movement of the probe in the axial direction than in the lateral direction.

In some examples, based on the probe safety margin 255, the control system may determine which, if any, critical structures 260 are more at risk of being affected by the ablative procedure than others. In FIGS. 2A and 2B, the critical structure 260A is more at risk than the critical structure 260B because the distance between the probe safety margin 255 and the critical structure 260A is less than the distance between the probe safety margin 255 and the critical structure 260B.

Referring back to FIG. 4B, after the optimization parameters have been determined, output parameters 136 can be identified based on the ablation zone(s). The output parameters 136 can be used to generate an optimized ablation treatment plan. The output parameters 136 can include one or more deployment poses (e.g., the deployment pose(s) 136A) for a medical device (e.g., medical device 104) which can include a delivery catheter and an ablation probe. As previously described, the deployment poses provide a location and orientation of the delivery catheter for delivery of the ablation probe towards the target. By determining optimal deployment poses, a minimum number of ablations can be performed to effectively treat a lesion (e.g., the anatomical target 108) and margin (e.g., the margin 110) thus reducing procedure time and improving procedure outcome. After deployment poses are identified, deployment paths (e.g., the deployment path(s) 136B) may be planned to help navigate the medical instrument into the desired deployment pose. The deployment paths and deployment poses can be used to create the optimized ablation treatment plan.

To determine each of the deployment poses, each of the ablation zones (e.g., the ablation zone(s) 240/340) determined from process 180 of method 170 may be utilized. Each ablation zone is created by the ablation probe positioned within an optimal treatment zone (e.g., the optimal treatment zone 235A). In some examples, the shape of the ablation zone is determined in part by the type and construction of the ablation probe. As illustrated in FIGS. 2A-3C, the ablation zone may be an ovoid. Accordingly, by determining a desired position of a transducer centered along a major axis of the ablation zone, the deployment orientation of the delivery catheter can be determined along a line projected from the major axis. In some examples, the distance the ablation probe may be extended is set at a default insertion distance or may be limited by mechanical constraints. Accordingly, the deployment position of the delivery catheter may be determined based on the default insertion distance.

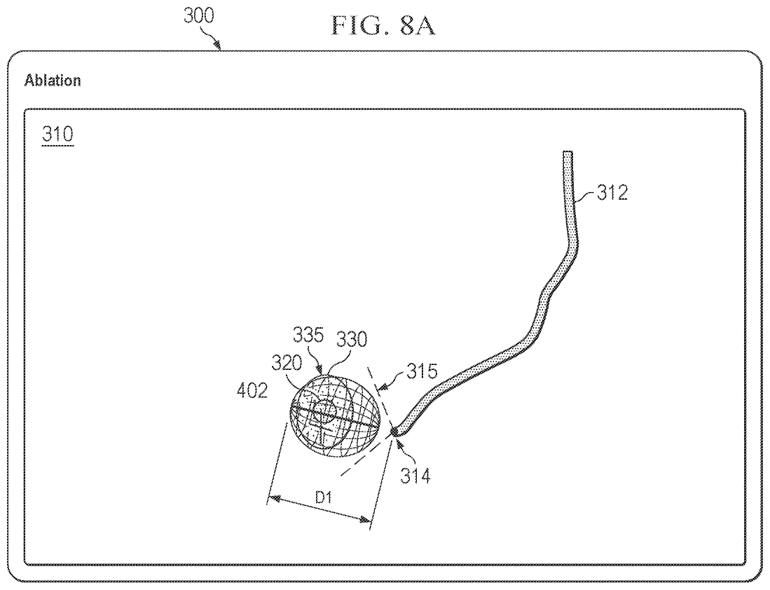

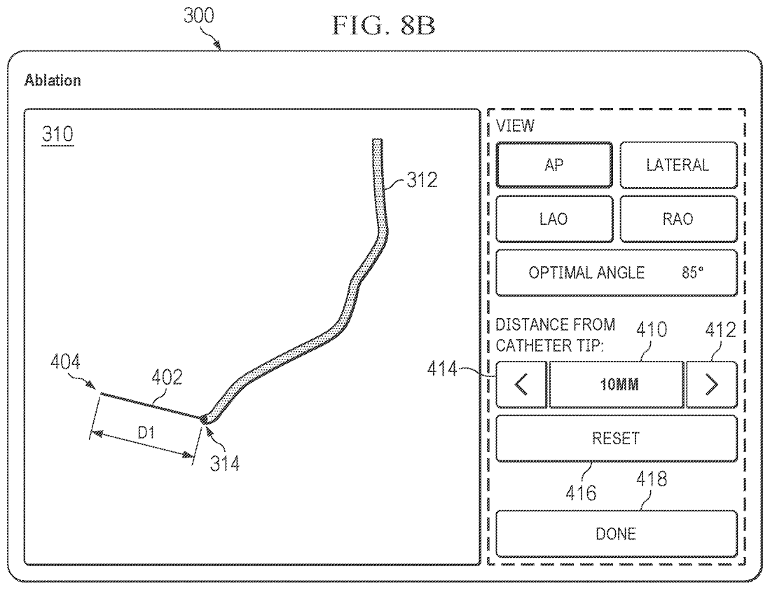

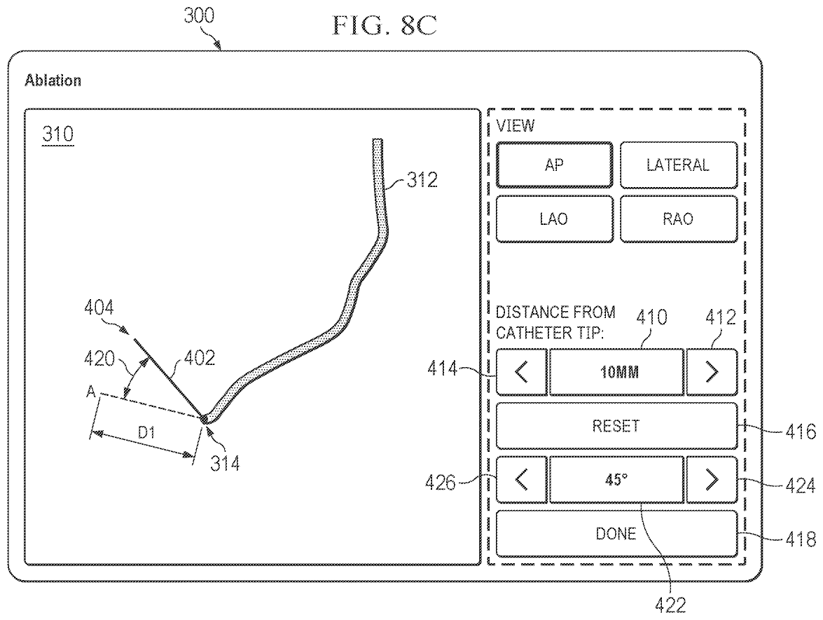

FIGS. 8A and 8B illustrate the GUI 300 in a planning or a navigation mode. An ablation probe 402 is shown as extending from the distal end 314 of the medical instrument 312 by an insertion distance D1, which may be shown in an icon 410 (FIG. 8B). The insertion distance may be measured from the distal end 314 of the medical instrument 312 to a distal end 404 of the ablation probe 402. In some examples, when the ablation probe 402 is extended from the medical instrument 312, the ablation probe 402 is extended by a known default insertion distance. The synthetic image of the ablation probe 402 may generate the insertion distance D1 based on the known default insertion distance. The default insertion distance may be 10 mm, as shown in FIG. 8A, but may be 5 mm, 15 mm, or any other desired distance. In some cases, the insertion distance D1 may be adjusted, such as adjustments made by a user via user inputs received at the GUI 300 and/or adjustments made automatically by the control system. Further details regarding FIGS. 8A and 8B will be discussed below.

In some examples, the available deployment poses for the medical instrument 312 in the deployment range 315 may orient the medical instrument 312 and/or a tool, such as an ablation probe, within the medical instrument 312 toward the target 320 in a direction that is perpendicular or substantially perpendicular to any critical structures near the target 320. This may help reduce the effects of a treatment, such as an ablation treatment, on the critical structures. In examples when more than one ablation treatment is needed to fully treat the clinical treatment zone 335, the medical instrument 312 may perform the treatments from the same deployment position within the deployment range 315 or from different deployment positions within the deployment range 315. In some examples, the different deployment positions may be located in different anatomical passageways 316. In some examples, a deployment location 318 (e.g., the deployment location 168) and/or a deployment orientation (e.g., the deployment orientation 170) of the medical instrument 312 is determined within the deployment range 315.

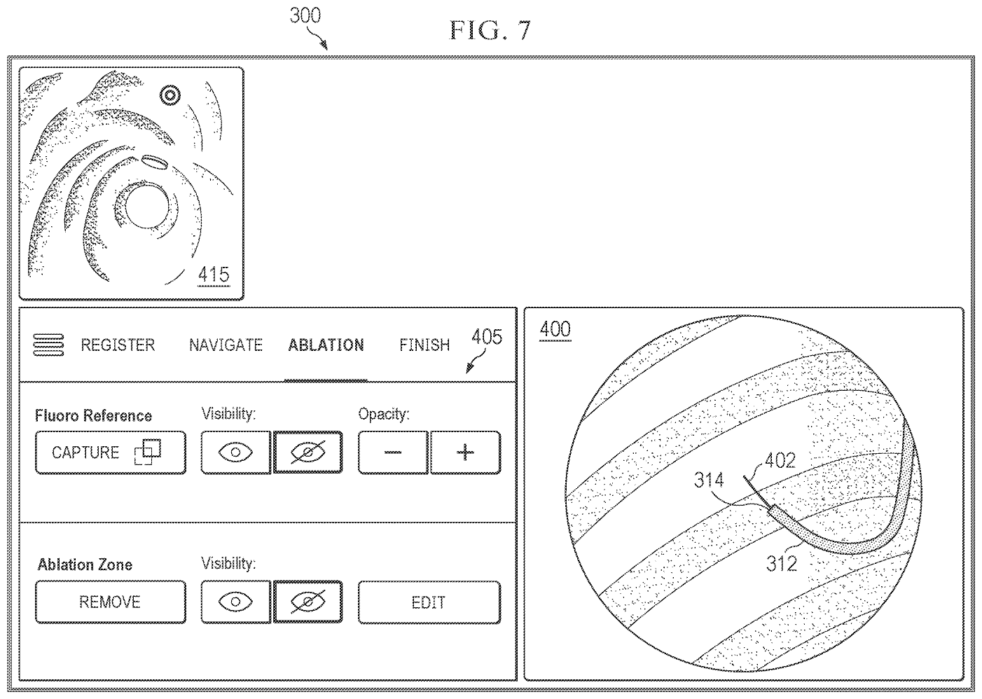

In some examples, a route of the medical instrument 312 through the patient anatomy to the deployment location 318 may be generated automatically by the control system. Additionally or alternatively, the control system may generate the route based on one or more user inputs. In some examples, the route may indicate a path along which the medical instrument 312 may be navigated into close proximity with the target 320. In some examples, the route may be stored in a control system (e.g., in a memory of a control system) and incorporated into the images displayed on the GUI 300. The view from the planned path for a current or selected location of the distal end 314 of the medical instrument 312 may be provided as a path view 415 in the GUI 300 (FIG. 7). The path view 415 may provide a synthetic view from within the anatomical passageways 316 and may include graphical markers (e.g., lines, arrows, or the like) indicating the path toward a target, such as the target 320. The path view 415 may optionally depict structures such as the target outside the walls of the anatomical passageways that would not be visible with an endoscopic camera positioned within the anatomical passageways.

In some examples, the patient anatomy may include more than one target. For example, as shown in FIG. 3C, the virtual navigation view 310 may display more than one target, such as the target 320, a target 321, and a target 322. Any other number of targets may be shown. The virtual navigation view 310 may also display a corresponding margin for each target that is displayed. For example, the clinical treatment zone 335, which includes the margin 330 corresponding to the target 320, may be displayed. A clinical treatment zone 333, which includes a margin 331 corresponding to the target 321, may additionally or alternatively be displayed. A clinical treatment zone 334, which includes a margin 332 corresponding to the target 322, may additionally or alternatively be displayed. Therefore, a clinical treatment zone may be displayed for each target 320, 321, 322. Deployment poses and deployment paths may be determined for each of the targets in a similar manner as previously described with reference to FIGS. 2A-5B.

FIG. 3C shows the medical instrument 312 positioned along a path toward the target 322. The target menu 370 indicates that the target 322 is selected. For example, the characteristics of the target 322 are shown in the target menu 370. Additionally or alternatively, when the target 322 is selected, the ablation zone 340 may be shown around the target 322, and critical structures 360 that are near the target 322 may be displayed. In some examples, the control system and/or the user may plan a path to one or more of the targets shown in the virtual navigation view 310. The paths may be stored at the control system, e.g., in a memory of the control system. The virtual navigation view 310 may display the path corresponding to a particular target when the particular target is selected by the user. For example, the path for the target 320 may be displayed when the user selected the target 320. In some examples, the virtual navigation view 310 may display the paths for each of the targets 320, 321, 322 simultaneously.

In some examples, potential airway exit points may be displayed in the virtual navigation view 310. The potential airway exit points may be displayed as numbered symbols, different colored symbols, and/or symbols with any other identifying feature. In examples when the potential airway exit points are displayed as numbered symbols, the numbering may be in priority of the exit point which provides the best ablation probe placement to the exit point that provides the worst ablation probe placement. The control system may determine the priority by determining how the ablation probe will function and/or how effective the ablation procedure will be when using each airway exit point. In some examples, the the numbering may be in priority of the exit point which provides the worst ablation probe placement to the exit point that provides the best ablation probe placement. The potential airway exit points may additionally or alternatively be displayed during an image-guided medical procedure.

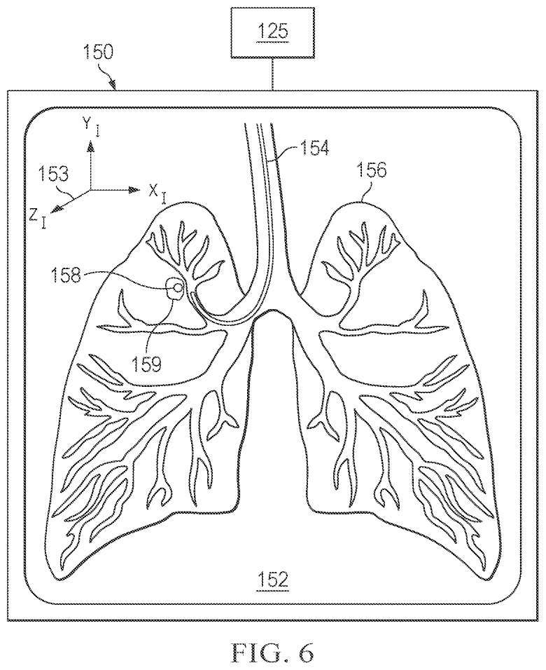

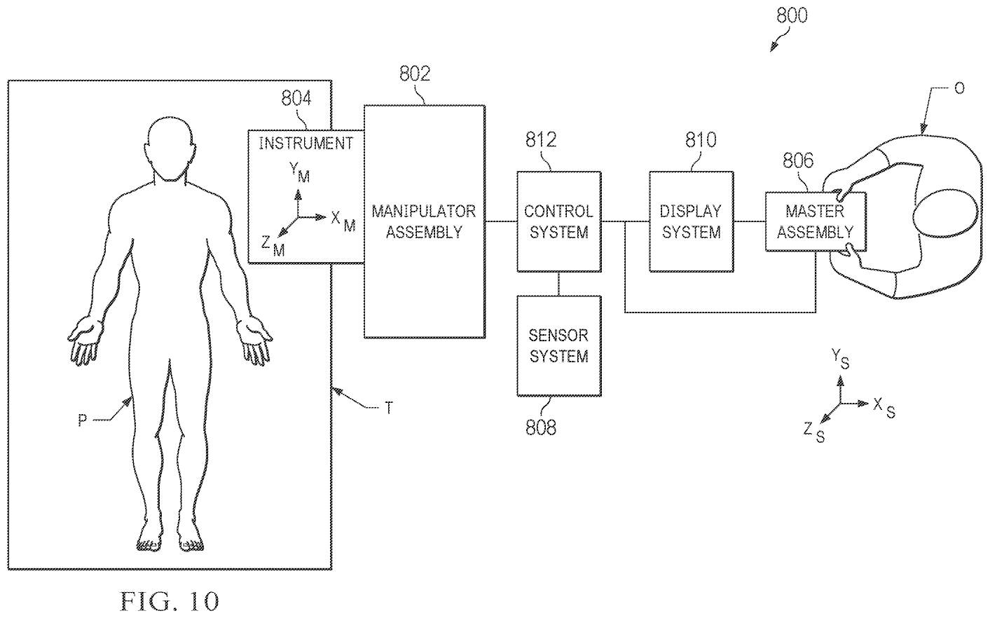

After treatment planning has been completed, the treatment plan may be exported and used during an image-guided medical procedure. With reference to FIG. 6, an image-guided medical procedure, which may be manually performed, robot-assisted, or otherwise teleoperated, may be conducted in which a display system 150 may display a virtual navigation image 152, which includes an image reference frame (X_{I}, Y_{I}, Z_{I}) 153. An elongate device, such as a medical instrument 154, which may be the medical instrument 104, may be registered (e.g., dynamically referenced) with an anatomic model 156 of a patient derived from pre-operative image data obtained, for example, from a computerized tomography (CT) scan. The anatomic model 156 may be the anatomic model 106 of FIG. 1. The anatomic model 156 may include a target 158, such as a lesion or nodule of interest, which the treatment plan and the procedure is intended to address (e.g., biopsy, treat, view, etc.). The target 158 may be the target 108. The target 158 may include a margin 159 (e.g., the margin 110) surrounding the target 158. In some examples, the virtual navigation image 152 may also or alternatively present a physician with a virtual image of the internal surgical site from a viewpoint of the medical instrument 154, such as from a distal tip of the medical instrument 154. In some examples, the display system 150 may also or alternatively present a real-time view from the distal tip of the medical instrument 154, such as when the medical instrument 154 includes an endoscope. In some examples, the medical instrument 154 may be manipulated by a robot-assisted manipulator controlled by the control system 125, or processing system, which includes one or more processors. An example of a robot-assisted medical system will be described further at FIG. 10. In some examples, an ablation probe may extend through a lumen of the medical instrument 154. In some examples, an ablation probe is the medical instrument 154.

Generating the virtual navigation image 152 involves the registration of the image reference frame (X_{I}, Y_{I}, Z_{I}) 153 to a surgical reference frame (X_{S}, Y_{S}, Z_{S}) of the anatomy and/or a medical instrument reference frame (X_{M}, Y_{M}, Z_{M}) of the medical instrument 154. This registration may rotate, translate, or otherwise manipulate by rigid or non-rigid transforms points associated with the segmented instrument shape from the image data and/or points associated with the shape data from a shape sensor disposed along a length of the medical instrument 154. This registration between the image and instrument reference frames may be achieved, for example, by using a point-based iterative closest point (ICP) technique as described in U.S. Provisional Pat. App. No. 62/205,440, filed on Aug. 14, 2015, entitled “Systems and Methods of Registration for Image-Guided Surgery” and in U.S. Provisional Pat. App. No. 62/205,433, filed on Aug. 14, 2015, entitled “Systems and Methods of Registration for Image-Guided Surgery,” which are incorporated by reference herein in their entireties. The registration may be achieved additionally or alternatively by another point cloud registration technique.

With reference to FIGS. 3A-3C, while previously described in the context of treatment planning, the GUI 300 may be used for intraoperative navigational guidance while performing the medical procedure. The virtual navigation view 310 may be generated by registering preoperative imaging data (and a subsequently constructed three-dimensional (3D) model, such as the model 156/106) to a current location of the medical instrument 312. The GUI 300 may also include one or more windows 305 that illustrate the target 320 in one or more cross-sections of the patient anatomy from various views (e.g., top view, side view, front view). The GUI 300 may also include a target menu 370 and a probe menu 380. Further details of the GUI 300 will be described below.

As shown in FIG. 3B, the GUI 300 may display the virtual navigation view 310 without the windows 305. This may allow the user to more closely analyze the details of the virtual navigation view 310, which may assist with planning the path for the medical instrument 312 and/or with navigating the medical instrument 312 along the path. In some examples, the virtual navigation view 310 shown in FIG. 3B may replace the virtual navigation view 310 in FIG. 3A. As shown in FIG. 3B, a distal end 314 of the medical instrument 312 may be navigated to a location near the target 320. The current shape of the medical instrument 312 and the location of the distal end 314 may be displayed in the virtual navigation view 310. The medical instrument 312 may be navigated by a user, a teleoperational control system (e.g., the control system 125), or a combination of manual and automatic inputs.

With reference to FIG. 7, the GUI 300 may also include an intraoperative external image 400 and an icon menu 405. The intraoperative external image 400 may be received at a control system (e.g., the control system 125) from an intraoperative external imaging system. In some examples, the intraoperative external imaging system may be a fluoroscopy imaging system than generates intraoperative fluoroscopy image data, although any suitable imaging technique, such as conventional CT or cone beam CT (“CBCT”) techniques, may be used without departing from the examples of the present disclosure. The intraoperative external imaging data may be received at a control system or other processing platform associated with the medical instrument 312. It is also contemplated that in some examples the shape data associated with the medical instrument 312 may be transferred to the imaging system, or both the shape data and the intraoperative external imaging data may be transferred to a common platform for processing. In this regard, registration of the shape data of the medical instrument 312 to the intraoperative external imaging data may be performed by the control system, by the imaging system, or by another platform in operable communication with the intraoperative external imaging system and the control system. In some examples, receiving the intraoperative external imaging data may include receiving one or more timestamps associated with the intraoperative external imaging data. A first timestamp may indicate the start time of the intraoperative external imaging and a second timestamp may additionally indicate a stop time of the intraoperative external imaging. Alternatively, a timestamp may be associated with each instance of intraoperative external imaging data. In order to ensure accurate correlation, a clock of the control system of the medical instrument 312 may be synchronized with a clock of the intraoperative external imaging system, and each instance of shape data may also be associated with a timestamp. In this regard, each timestamped instance of intraoperative external imaging data may be paired with a correspondingly timestamped instance of shape data.

In some examples, an ablation may be performed when the medical instrument 312 reaches the deployment location 318. The ablation may be performed by an ablation probe 402, which may be deployed through a lumen of the medical instrument 312. Alternatively, the ablation probe 402 may be inserted into the anatomical passageways 316 independently of the medical instrument 312. In some examples, the ablation probe 402 may be independently manipulatable with respect to the medical instrument 312. For example, the ablation probe 402 may be articulated independently of the medical instrument 312. The ablation probe 402 may be coupled to one or more electrical wires or optical fibers for activating the ablation probe 402, modulating its output, capturing return signals, and/or the like.

In some examples, the anatomical passageways 316 may be hidden from view and removed from the virtual navigation view 310 displayed in the GUI 300. The control system may receive a user input selecting a “hide airways” icon (not shown) on the GUI 300, or the control system may hide the anatomical passageways 316 in response to other triggers such as beginning the ablation. With the anatomical passageways 316 hidden, the user may more easily see the medical instrument 312 in the virtual navigation view 310. This may allow for the user to more clearly visualize the orientation of the medical instrument 312 in the virtual navigation view 310. This may additionally or alternatively allow for more refined and accurate adjustments to the shape of the medical instrument 312 to be made, which will be discussed in further detail below. Additionally or alternatively, the anatomical passageways 316 may be made transparent with outlines of the anatomical passageways 316 still visible in the virtual navigation view 310. In some examples, the target 320 may similarly be hidden and removed from the virtual navigation view 310. In some examples, the critical structures 360 may remain visible in the virtual navigation view 310 while the anatomical passageways 316 are hidden. In some examples, the critical structures 360 may be hidden from view and removed from the virtual navigation view 310. The control system may receive a user input selecting a “hide critical structures” icon (not shown) on the GUI 300, or the control system may hide the critical structures 360 in response to other triggers such as beginning the ablation.

With reference to FIGS. 8A and 8B, the GUI 300 may be displayed in a planning or a navigation mode. In the planning mode, the position and orientation of the medical instrument 312 may be determined as an optimal deployment pose or selected deployment pose using processes previously described above. In the navigation mode, the position and orientation of the medical instrument 312 may be determined using localization sensors coupled to the medical instrument 312, such as fiber optic shape sensors, electromagnetic sensors, and/or the like, as will be described in further detail below. Some exemplary details regarding using the GUI 300 in the navigation mode will now be discussed. In some examples, a synthetic image of the ablation probe 402 may be shown in the virtual navigation view 310 of the GUI 300. In FIG. 8A, the ablation probe 402 is shown as extending from the distal end 314 of the medical instrument 312 by an insertion distance D1, which may be shown in an icon 410 (FIG. 8B). The insertion distance may be measured from the distal end 314 of the medical instrument 312 to a distal end 404 of the ablation probe 402. In some examples, when the ablation probe 402 is extended from the medical instrument 312, the ablation probe 402 is extended by a known default insertion distance. The synthetic image of the ablation probe 402 may generate the insertion distance D1 based on the known default insertion distance. The default insertion distance may be 10 mm, as shown in FIG. 8A, but may be 5 mm, 15 mm, or any other desired distance. In some cases, the insertion distance D1 may be adjusted.

As shown in FIG. 8B, the GUI 300 may include an increase icon 412 and a decrease icon 414. The insertion distance D1 may increase when the control system receives a user input selecting the increase icon 412. Similarly, the insertion distance D1 may decrease when the control system receives a user input selecting the decrease icon 414. The insertion distance D1 may be increased or decreased in increments of 1 mm but may be increased or decreased in increments of 0.5 mm, 2 mm, 3 mm, or any other desired distance. To reset the insertion distance D1 to the default insertion distance, an icon 416 may be selected. In some examples, the ablation probe 402 be hidden and removed from the virtual navigation view 310 as discussed above with respect to FIG. 7. Any adjustments made to the insertion distance D1 and/or any other adjustments made to the ablation probe 402 may be confirmed when the control system receives a user input selecting a “Done” icon 418. The confirmed adjustments may be revisited and further adjusted at any time.

In some examples, the control system may receive a user input selecting (e.g., touching or clicking) the distal end 314 of the medical instrument 312 and a far edge of the target 320 to measure the distance between the distal end 314 of the medical instrument and the far edge of the target 320. This distance may be displayed in the GUI 300, such as in the virtual navigation view 310, or in any other location in the GUI 300. Additionally or alternatively, the control system may determine the distance between the distal end 314 of the medical instrument 312 and the far edge of the target 320 using imaging analysis.

As shown in FIG. 8C, the ablation probe 402 is shown as bending away from a longitudinal axis A of the medical instrument 312 by an angle 420. The angle 420 may be measured from the longitudinal axis A of the medical instrument 312 to the ablation probe 402. The degree of the angle 420 may be shown in an icon 422 of the GUI 300. As shown in FIG. 8C, the GUI 300 may further include an increase icon 424 and a decrease icon 426. The angle 420 may increase when the control system receives a user input selecting the increase icon 424. Similarly, the angle 420 may decrease when the control system receives a user input selecting the decrease icon 426. The angle 420 may be increased or decreased in increments of 1° (i.e., one degree) but may be increased or decreased in increments of 0.5°, 2°, 3°, or any other desired amount of degrees. As shown in FIG. 8C, the angle 420 may be 30°. In some examples, the ablation probe 402 may bend to any angle within the deployment range 315.

In some examples, when the ablation probe 402 is bending away from the longitudinal axis A, the ablation probe 402 may be shown within an image of an anatomical passageway, such as one of the anatomical passageways 316. This may help illustrate that the ablation probe 402 may remain within an anatomical passageway even when bent away from the longitudinal axis A. Any adjustments made to the angle 420 and/or any other adjustments made to the ablation probe 402 may be confirmed when the control system receives a user input selecting the “Done” icon 418. The confirmed adjustments may be revisited and further adjusted at any time.

In some examples, the virtual navigation view 310 may display the medical instrument 312 at any position with a deployment location 318 within the deployment range 315 where the maximum bend radius of the medical instrument 312 is below a threshold bend radius. The control system may generate a mechanical model of the medical instrument 312 within in the model of the anatomical passageways 316. In some examples, as the bend radius of the medical instrument 312 changes, the portions of the medical instrument 312 that are bent may change color or may change in any other graphical manner. For example, as a portion of the medical instrument 312 bends, the portion may change from a green color to a red color as the bend radius increases.

In some examples, the control system may receive a user input at the GUI 300 switching between different workflow modes/steps displayed on the GUI 300. For example, the workflow modes/steps may be switched between a needle insertion workflow, an ablation probe insertion workflow, and a medical instrument retraction workflow. In some examples, more than one workflow may be displayed on the GUI 300 at the same time. For example, the workflows may be displayed in separate windows of the GUI 300 or as a picture-in-picture.

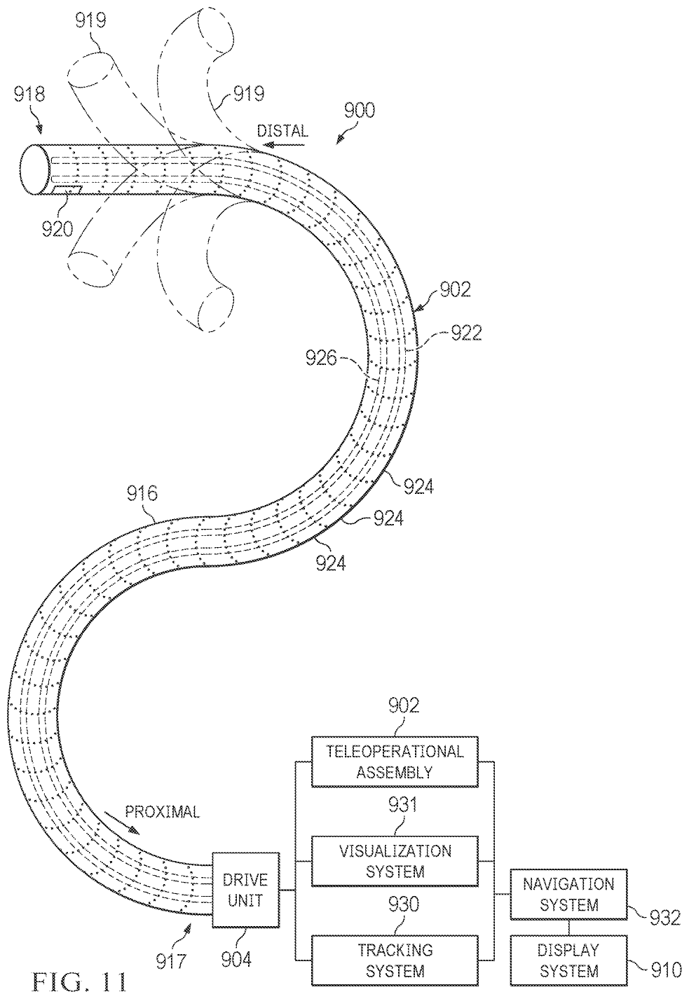

In some examples, the displayed bend angle of the medical instrument 312 may be adjusted or altered due to the shape of the medical instrument 312 being affected by stiffness changes when the ablation probe 402 is installed and inserted through the medical instrument 312. The shape of the medical instrument 312 may be measured with fiber optic shape sensors and/or any localization sensor(s), such as an electromagnetic sensor. Additional details regarding measuring the shape of the medical instrument 312 are discussed below with respect to FIG. 11. In some examples, the control system may determine the stiffness of the medical instrument 312 by measuring the force exerted on pull wires in the medical instrument 312 as the medical instrument 312 is bent. In some examples, as the stiffness of the medical instrument 312 changes, the portions of the medical instrument 312 that have a changing stiffness may change color or may change in any other graphical manner. For example, as a portion of the medical instrument 312 becomes stiffer, the portion may change from a green color to a red color as the stiffness increases.

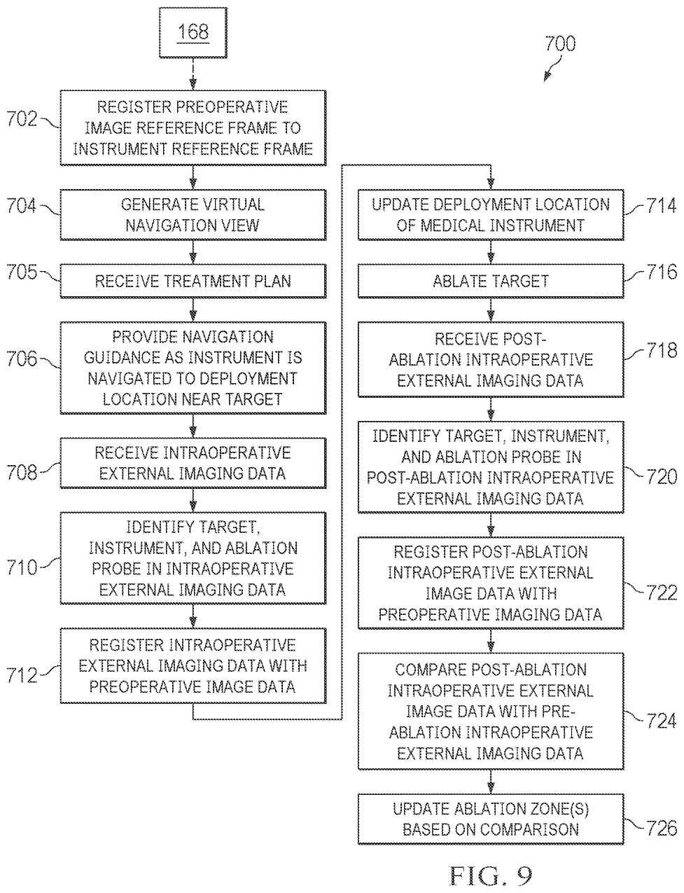

With reference now to FIG. 9, FIG. 9 includes a flowchart illustrating an optional method 700 for planning a medical procedure, such as an ablation procedure, and navigating a medical instrument during the medical procedure. The method 700 is illustrated as a set of operations or processes 702-726. The method 700 may optionally be performed after the one or more deployment parameters are determined based on the clinical treatment zone (e.g., the clinical treatment zone 235) at the process 168. The processes 702-726 may be performed in the same or in a different order than the order shown in FIG. 9. One or more of the illustrated processes may be omitted in some examples of the method 700. Additionally, one or more processes that are not expressly illustrated in the flowchart may be included before, after, in between, or as part of the illustrated processes. In some examples, one or more of the processes of the flowchart may be implemented, at least in part, by a control system executing code stored on non-transitory, tangible, machine-readable media that when run by one or more processors (e.g., the processors of a control system) may cause the one or more processors to perform one or more of the processes.

To provide accurate navigation through the anatomical passageways, the reference frame 120 of the preoperative imaging data (and subsequently constructed 3D model) may be registered to a reference frame of the medical instrument 312 at a process 702. For example, a shape sensor (e.g., a fiber optic shape sensor and/or one or more position sensors) disposed along a length of the medical instrument 312 may be used to provide real-time shape data (e.g., information regarding a shape of the medical instrument 312 and/or a position of one or more points along the length of the medical instrument 312). This shape data may be utilized to register the medical instrument 312 to the 3D model constructed from the preoperative imaging data and to track a location of the medical instrument 312 during use. Upon successful registration, a process 704 may include generating a virtual navigation view (e.g., the virtual navigation view 310 of FIGS. 3A-3C).

At a process 705, a treatment plan is received by the control system. For example, the ablation treatment plan discussed above may be received by the control system 125. The treatment plan may be used during an image-guided medical procedure.

At a process 706, navigation guidance is provided as the medical instrument 312 is navigated through the anatomical passageways 316 to a predetermined deployment location in proximity to the target 320. The predetermined deployment location may be the deployment location 318 determined in the treatment planning process discussed above. In some examples, as shown in FIG. 3B, the distal end 314 of the medical instrument 312 may be navigated to the deployment location 318 near the target 320. Navigation may be performed manually by a user with provided navigation guidance, automatically by the control system, or via a combination of both.

In some examples, the navigation guidance may be provided by overlaying a fluoroscopic reference of a pilot hole and/or biopsy markers over a measured shape of the medical instrument 312. This may allow the control system and/or the user to adjust the position of the distal end 314 of the medical instrument 312 to be parallel to the fluoroscopic reference or coincident with the biopsy marker. In some examples, the measured shape of the medical instrument 312 may be displayed as a shape overlayed on the fluoroscopic reference.

With the medical instrument 312 positioned at the deployment location 318, preferably in close proximity to the target 320, an intraoperative external imaging scan may be performed. At a process 708, intraoperative external imaging data may be received at a control system from an intraoperative external imaging system. In some examples, the intraoperative external imaging system may be a fluoroscopy imaging system than generates intraoperative fluoroscopy image data, although any suitable imaging technique, such as conventional CT or cone beam CT (“CBCT”) techniques, may be used without departing from the examples of the present disclosure. The intraoperative external imaging data may be displayed in the GUI 300 as the intraoperative external image 400 (e.g., a fluoroscopic image). At a process 710, the medical instrument 312 and the ablation probe 402 may be identified in the intraoperative external image 400. The identification may be made by the control system (e.g., using image processing) and/or by an operator.

In order to register the fluoroscopic image 400 to the medical instrument 312, while the intraoperative external imaging is performed, at a process 712, shape data from the medical instrument 312 captured during the intraoperative external imaging process 708 may be received. The shape data may be captured for only a brief period of time or may be captured during the whole image capture period of the intraoperative external imaging process.

At a process 714, the deployment location 318 of the medical instrument 312 is updated as needed. The deployment location 318 may be updated based on the intraoperative external imaging data. The position of the ablation probe 402 may also be adjusted as needed based on the intraoperative external imaging data. The updated deployment location may be within the deployment range 315.

At a process 716, the target 320 is ablated. The control system may perform and/or assist with the ablation procedure. For example, the amount of power illustrated by the power icon 382 of FIG. 3B is supplied to the ablation probe 402 for the amount of time illustrated by the time icon 384 of FIG. 3B. If more than one ablation zone is needed to fully cover the clinical treatment zone 335, an ablation treatment is applied for each ablation zone according to the treatment plan. At a process 718, intraoperative external imaging data may be received at a control system from an intraoperative external imaging system after the ablation treatment is completed. In some examples, the intraoperative external imaging system may be a fluoroscopy imaging system than generates intraoperative fluoroscopy image data, although any suitable imaging technique, such as conventional CT or cone beam CT (“CBCT”) techniques, may be used without departing from the examples of the present disclosure. The intraoperative external imaging data may be displayed in the GUI 300. For example, the pre-ablation intraoperative external image 400 (e.g., a fluoroscopic image) shown in FIG. 7 may be replaced with a post-ablation intraoperative external image. Each ablation zone in the treatment plan may be displayed in the post-ablation intraoperative external image. In some examples, both the pre-ablation intraoperative external image 400 and the post-ablation intraoperative external image may be displayed in the GUI 300 at the same time. The two images may displayed in different windows of the GUI 300, as a picture-in-picture in the same window of the GUI 300, or in any other arrangement in the GUI 300. At a process 720, the medical instrument 312 and the ablation probe 402 may be identified in the post-ablation intraoperative external image. The identification may be made by the control system (e.g., using image processing) and/or by an operator.

At a process 722, the post-ablation intraoperative image data is registered with the preoperative imaging data. As discussed above at the process 712, the pre-ablation intraoperative image data may be registered with the preoperative imaging data. In a similar manner to the manner described above at process 712, the post-ablation intraoperative image data may be registered with the preoperative imaging data. For example, while the post-ablation intraoperative external imaging is performed, shape data from the medical instrument 312 captured during the post-ablation intraoperative external imaging process 718 may be received. The shape data may be captured for only a brief period of time or may be captured during the whole image capture period of the post-ablation intraoperative external imaging process. As discussed above with respect to the process 712, in some examples, each timestamped instance of post-ablation intraoperative external imaging data may be paired with a correspondingly timestamped instance of shape data from the medical instrument 312.

At a process 724, the post-ablation intraoperative external imaging data is compared with the pre-ablation intraoperative external imaging data. The comparison may be performed by the control system and/or the operator. In some examples, the control system may use image analysis and/or an imaging processor to compare the image data. During the comparison, the control system may determine whether the ablation zone 340 actually covered the entire clinical treatment zone 335. If the entire clinical treatment zone 335 was not covered by the ablation zone 340, then one or more additional ablation treatments may be needed to fully ablate the clinical treatment zone 335. For example, the actual ablation zone 340 that was applied during the ablation treatment may have been changed (e.g., in size and/or shape) from the planned ablation zone. The change in the ablation zone 340 may be the result of one or more factors, such as a density of the target 320, a density of nearby blood vessels, a size of nearby blood vessels, a location of one or more critical structures 360, or any other similar factor.

In some examples, the control system may determine a proximity between a current position of the target 320 and a current position of the medical instrument 312 when comparing the post-ablation intraoperative external imaging data with the pre-ablation intraoperative external imaging data. The control system may additionally or alternatively determine a proximity between a current position of the target 320 and a current position of one or more of the critical structures 360 when comparing the post-ablation intraoperative external imaging data with the pre-ablation intraoperative external imaging data. In some examples, a position of the critical structure 360 in the post-ablation intraoperative external imaging data may have shifted relative to a position of the critical structure 360 in the pre-ablation intraoperative external imaging data. When comparing the post-ablation intraoperative external imaging data with the pre-ablation intraoperative external imaging data, the control system may determine a proximity between a current position of the target 320 and a current position of the critical structure 360 in the post-ablation intraoperative external imaging data. In some examples, the control system may determine a proximity between a current position of the target 320 and a current position of the one or more anatomical passageways 316 in the post-ablation intraoperative external imaging data when comparing the post-ablation intraoperative external imaging data with the pre-ablation intraoperative external imaging data.

At a process 726, the ablation zone 340 and any other necessary ablation zones are updated based on the comparison between the post-ablation intraoperative external imaging data and the pre-ablation intraoperative external imaging data. One or more additional ablation treatments may be performed as needed based on the updated ablation zone(s) 340. An updated deployment location may be determined for each updated ablation zone. The updated deployment location may be determined in a similar manner to the manner described at the process 714. In some examples, the medical instrument 312 may be repositioned to a different anatomical passageway 316 to reach the updated deployment location.

In some examples, the GUI 300 may include one or more windows used to display a preview of an ablation procedure for one or more selected deployment locations. This may allow the control system and/or the user to determine the effectiveness of an ablation procedure if the ablation probe 402 is deployed from the specific, selected deployment location. The ablation simulation may be provided based on tissue type (e.g., lung, airway). In some examples, the ablation simulation may be a three-dimensional simulation. Additionally or alternatively, ablation simulation may be a two-dimensional simulation.

In some examples, the components discussed above may be part of a robotic-assisted system as described in further detail below. The robotic-assisted system may be suitable for use in, for example, surgical, robotic-assisted surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general robotic-assisted, or robotic medical systems.

As shown in FIG. 10, a medical system 800 generally includes a manipulator assembly 802 for operating a medical instrument 804 (e.g., the medical instrument 104) in performing various procedures on a patient P positioned on a table T. The manipulator assembly 802 may be robotic-assisted, non-robotic-assisted, or a hybrid robotic-assisted and non-robotic-assisted assembly with select degrees of freedom of motion that may be motorized and/or robotic-assisted and select degrees of freedom of motion that may be non-motorized and/or non-robotic-assisted. The medical system 800 may further include a master assembly 806, which generally includes one or more control devices for controlling manipulator assembly 802. Manipulator assembly 802 supports medical instrument 804 and may optionally include a plurality of actuators or motors that drive inputs on medical instrument 804 in response to commands from a control system 812. The actuators may optionally include drive systems that when coupled to medical instrument 804 may advance medical instrument 804 into a naturally or surgically created anatomic orifice.

Medical system 800 also includes a display system 810 for displaying an image or representation of the surgical site and medical instrument 804 generated by sub-systems of sensor system 808. Display system 810 and master assembly 806 may be oriented so operator O can control medical instrument 804 and master assembly 806 with the perception of telepresence. Additional information regarding the medical system 800 and the medical instrument 804 may be found in International Application Publication No. WO 2018/195216, filed on Apr. 18, 2018, entitled “Graphical User Interface for Monitoring an Image-Guided Procedure,” which is incorporated by reference herein in its entirety.

In some examples, medical instrument 804 may include components of an imaging system (discussed in more detail below), which may include an imaging scope assembly or imaging instrument that records a concurrent or real-time image of a surgical site and provides the image to the operator or operator O through one or more displays of medical system 800, such as one or more displays of display system 810. The concurrent image may be, for example, a two or three-dimensional image captured by an imaging instrument positioned within the surgical site. In some examples, the imaging system includes endoscopic imaging instrument components that may be integrally or removably coupled to medical instrument 804. However, in some examples, a separate endoscope, attached to a separate manipulator assembly may be used with medical instrument 804 to image the surgical site. In some examples, as described in detail below, the imaging instrument alone or in combination with other components of the medical instrument 804 may include one or more mechanisms for cleaning one or more lenses of the imaging instrument when the one or more lenses become partially and/or fully obscured by fluids and/or other materials encountered by the distal end of the imaging instrument. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Application Publication No. WO/2016/025465, filed on Aug. 11, 2016, entitled “Systems and Methods for Cleaning an Endoscopic Instrument”; U.S. patent application Ser. No. 15/508,923, filed on Mar. 5, 2017, entitled “Devices, Systems, and Methods Using Mating Catheter Tips and Tools”; and U.S. patent application Ser. No. 15/503,589, filed Feb. 13, 2017, entitled “Systems and Methods for Cleaning an Endoscopic Instrument,” each of which is incorporated by reference herein in its entirety. The imaging system may be implemented as hardware, firmware, software or a combination thereof which interact with or are otherwise executed by one or more computer processors, which may include the processors of the control system 812.

Control system 812 includes at least one memory and at least one computer processor (not shown) for effecting control between medical instrument 804, master assembly 806, sensor system 808, and display system 810. Control system 812 also includes programmed instructions (e.g., a non-transitory machine-readable medium storing the instructions) to implement some or all of the methods described in accordance with aspects disclosed herein, including instructions for providing information to display system 810.

FIG. 11 is a simplified diagram of a medical instrument system 900 according to some examples. Medical instrument system 900 includes elongate device 902, such as a flexible catheter (e.g., the medical instrument 312), coupled to a drive unit 904. Elongate device 902 includes a flexible body 916 having proximal end 917 and distal end or tip portion 918. Medical instrument system 900 further includes a tracking system 930 for determining the position, orientation, speed, velocity, pose, and/or shape of distal end 918 and/or of one or more segments 924 along flexible body 916 using one or more sensors and/or imaging devices as described in further detail below.

Tracking system 930 may optionally track distal end 918 and/or one or more of the segments 924 using a shape sensor 922. Shape sensor 922 may optionally include an optical fiber aligned with flexible body 916 (e.g., provided within an interior channel (not shown) or mounted externally). The optical fiber of shape sensor 922 forms a fiber optic bend sensor for determining the shape of flexible body 916. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389, filed on Jul. 13, 2005, entitled “Fiber Optic Position and Shape Sensing Device and Method Relating Thereto”; U.S. patent application Ser. No. 12/047,056, filed on Jul. 16, 2004, entitled “Fiber-Optic Shape and Relative Position Sensing”; and U.S. Pat. No. 6,389,187, filed on Jun. 17, 1998, entitled “Optical Fibre Bend Sensor”, each of which is incorporated by reference herein in its entirety. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 916 can be used to reconstruct the shape of flexible body 916 over the interval of time. In some examples, tracking system 930 may optionally and/or additionally track distal end 918 using a position sensor system 920. Position sensor system 920 may be a component of an EM sensor system with position sensor system 920 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 920 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732, filed on Aug. 11, 1999, entitled “Six-Degree of Freedom Tracking System Having a Passive Transponder on the Object Being Tracked”, which is incorporated by reference herein in its entirety.

Flexible body 916 includes a channel 921 sized and shaped to receive a medical instrument 926. Further description of a medical instrument received by a flexible body is provided in U.S. Provisional Patent Application No. 63/077,059, filed on Sep. 11, 2020, entitled “Systems for Coupling and Storing an Imaging Instrument”, which is incorporated by reference herein in its entirety.

Flexible body 916 may also house cables, linkages, or other steering controls (not shown) that extend between drive unit 904 and distal end 918 to controllably bend distal end 918 as shown, for example, by broken dashed line depictions 919 of distal end 918. In some examples, at least four cables are used to provide independent “up-down” steering to control a pitch of distal end 918 and “left-right” steering to control a yaw of distal end 918. Steerable elongate devices are described in detail in U.S. patent application Ser. No. 13/274,208, filed on Oct. 14, 2011, entitled “Catheter with Removable Vision Probe”, which is incorporated by reference herein in its entirety.

The information from tracking system 930 may be sent to a navigation system 932 where it is combined with information from image processing system 931 and/or the preoperatively obtained models to provide the operator with real-time position information. In some examples, the real-time position information may be displayed on display system 810 of FIG. 10 for use in the control of medical instrument system 900. In some examples, control system 812 of FIG. 10 may utilize the position information as feedback for positioning medical instrument system 900. Various systems for using fiber optic sensors to register and display a surgical instrument with surgical images are provided in U.S. patent application Ser. No. 13/107,562, filed on May 13, 2011, entitled “Medical System Providing Dynamic Registration of a Model of an Anatomic Structure for Image-Guided Surgery,” which is incorporated by reference herein in its entirety.

In some examples, medical instrument system 900 may be robotic-assisted within medical system 800 of FIG. 10. In some examples, manipulator assembly 802 of FIG. 10 may be replaced by direct operator control. In some examples, the direct operator control may include various handles and operator interfaces for hand-held operation of the instrument.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Further, although some of the examples presented in this disclosure discuss robotic-assisted systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium (e.g., a non-transitory storage medium) or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain example examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A medical system comprising:
a control system configured to:
receive first imaging data of a patient anatomy;
identify an anatomical target in the patient anatomy;
generate a treatment zone having a first axis, wherein the treatment zone includes the anatomical target; and
determine a deployment position of an elongate device configured to receive a medical instrument for treatment of the anatomical target, wherein the deployment position is aligned with the first axis of the treatment zone.

2. The medical system of claim 1, wherein the deployment position includes a deployment location or a deployment orientation.

3. The medical system of claim 1, wherein the control system is further configured to;
identify one or more critical structures in the patient anatomy; and
alter the treatment zone based on the one or more critical structures.

4-8. (canceled)

9. The medical system of claim 1, wherein the control system is further configured to determine a deployment range of the elongate device.

10-13. (canceled)

14. The medical system of claim 9, wherein an orientation of the deployment range is determined based on a major axis of the anatomical target.

15. The medical system of claim 1, wherein the control system is further configured to determine at least one ablation zone, wherein the at least one ablation zone covers the treatment zone.

16. The medical system of claim 15, wherein determining the at least one ablation zone further includes minimizing a size of an ablation region of the at least one ablation zone beyond the treatment zone.

17-23. (canceled)

24. The medical system of claim 16, wherein the control system is further configured to receive a user input adjusting a size of the at least one ablation zone.

25. The medical system of claim 16, wherein the control system is further configured to adjust ablation parameters based on a proximity of the at least one ablation zone to one or more critical structures.

26. (canceled)

27. The medical system of claim 1, wherein the control system is further configured to display an image of the treatment zone and an image of the anatomical target via a display system including a graphical user interface.

28. (canceled)

29. (canceled)

30. A method comprising:
receiving first imaging data of a patient anatomy;
receiving information identifying an anatomical target in the patient anatomy;
generating a treatment zone including the anatomical target; and
determining a first axis of the treatment zone to identify a deployment location of an elongate device, wherein the deployment location is aligned with the first axis of the treatment zone.

31-35. (canceled)

36. The method of claim 30, further comprising adjusting the treatment zone based on an identified critical structure in the patient anatomy.

37. The method of claim 36, wherein adjusting the treatment zone includes shifting the first axis of the treatment zone laterally from a major axis of the anatomical target, and wherein the shifted first axis is parallel to the major axis.

38-43. (canceled)

44. The method of claim 30, further comprising determining a deployment location for a medical instrument configured to extend within the elongate device, wherein at the deployment location a longitudinal axis of the medical instrument is aligned with the first axis of the treatment zone.

45. (canceled)

46. The method of claim 30, further comprising:
receiving second imaging data of a patient anatomy; and
generating a second treatment zone based on the second imaging data.

47-59. (canceled)

60. A medical system comprising:
a display system;
an elongate device;
a medical instrument configured to extend within the elongate device; and
a control system communicatively coupled to the display system, the control system configured to:
display a graphical user interface via the display system, the graphical user interface including a virtual navigation view;
display an image of the elongate device in the virtual navigation view;
display an anatomical target in the virtual navigation view;
generate a treatment zone having a first axis, wherein the treatment zone includes the anatomical target; and
determine a deployment location of the elongate device, wherein the deployment location is aligned with the first axis of the treatment zone.

61. The medical system of claim 60, wherein the control system is further configured to display at least one of the treatment zone, the deployment location, or a deployment range of the medical instrument in the virtual navigation view.

62-64. (canceled)

65. The medical system of claim 60, wherein the control system is further configured to;
determine at least one ablation zone that covers the treatment zone; and
display the at least one ablation zone in the virtual navigation view.

66. (canceled)

67. (canceled)

68. The medical system of claim 65, wherein the control system is further configured to receive a user input adjusting one or more ablation parameters based on;
a proximity of the at least one ablation zone to one or more critical structures, or
a proximity of the at least one ablation zone to one or more anatomical passageways in close proximity to the anatomical target.

69. (canceled)

70. The medical system of claim 60, wherein the control system is further configured to determine a safety margin surrounding the treatment zone, wherein the safety margin is sized and shaped based on potential movement of a position of the medical instrument.

71. (canceled)
